(19) United States
(12) Patent Application Publication
(10) Pub. No.: US 2024/0085441 A1
(43) Pub. Date: Mar. 14, 2024

(54) AIR GAP PAPER ANALYTICAL DEVICE AND FABRICATION

(71) Applicant: UNIVERSITY OF NOTRE DAME DU LAC, South Bend, IN (US)

(72) Inventors: Rachel Marie ROLLER, South Bend, IN (US); Marya LIEBERMAN, South Bend, IN (US)

(21) Appl. No.: 18/453,116

(22) Filed: Aug. 21, 2023

Related U.S. Application Data
(60) Provisional application No. 63/373,059, filed on Aug. 21, 2022.

(51) Int. Cl. G01N 33/94; B01L 3/00

(52) U.S. Cl. CPC G01N 33/94; B01L 3/5023; B01L 2200/12; B01L 2200/16; B01L 2300/021; B01L 2300/0681; B01L 2300/069

(57) ABSTRACT

A paper analytical device for detecting an analyte in a sample, and methods of fabricating and using the same. A paper analytical device may include laminated strips of a porous hydrophilic substrate and an adhesive layer. The laminated strips may be disposed on a hydrophobic backing with an air gap barrier between each laminated strip that is about 1 mm to about 5 mm in width and extends along the entire length of the laminated strip. A paper analytical device also may include one or more assay regions having one or more reagents for detecting an analyte, and sample deposition areas for receiving a test sample.

### RELATED APPLICATIONS

This application claims priority under 35 U.S.C. § 119(e) to U.S. Provisional Patent Application No. 63/373,059, filed Aug. 21, 2022, which is incorporated herein by reference.

### STATEMENT REGARDING FEDERAL FUNDING

This invention was made with government support under grants EAGER-ISN CMMI 1842369 and IIP2016516 awarded by National Science Foundation. The government has certain rights in the invention.

### BACKGROUND OF THE INVENTION

Microfluidic paper analytical devices, or gADs, are a promising platform for point-of-use testing because of their low cost, portability, rapid results, and ease of use. A landmark article by the Whitesides group in 2007 sparked an explosion of research interest in gADs for applications as far-reaching as environmental monitoring, chemical education, pharmaceutical screening, and point-of-care diagnostics. Despite the large number of published academic articles on gADs, however, very few gADs have made it out of the academic laboratory and into the real world. One key reason for the difficulty of this “benchtop-to-bedside” transition is the lack of scalable fabrication methods for paper microfluidics.

Fabrication methods have been an ongoing challenge for the field of paper microfluidics since its inception. Early gADs relied on photolithography, which requires expensive materials, specialized equipment, and trained personnel. Many of the laboratory-based methods developed since, such as silanization, chemical vapor deposition, and plasma treatment, suffer from the same issues. Others, like hand cutting, hand drawing, hand folding, plotting, craft cutting, 3D printing, stamping, wax dipping, spraying, and screen printing, boast low cost and require little equipment, but still suffer from low throughput and high hands-on labor. When wax printing was applied to pPAD fabrication, it was soon heralded as the method of choice for pPAD fabrication due to its reasonable cost, moderate throughput, simple operation, and ease of prototyping. In 2016, however, Xerox discontinued its line of solid-ink printers, and no other company has picked up the technology. Other printer-based methods, like laser printing, inkjet printing, and recently, thermal transfer printing, have been explored as potential alternatives to wax printing, but remain most suitable for mid-scale applications requiring only moderate throughput.

Accordingly, there is a need for a simple, robust, cost effective, and easily produced paper analytical device with high sensitivity for the target analyte. The present invention satisfies these needs.

### SUMMARY OF THE INVENTION

The air-gap PAD sprang from the need to find a scalable alternative to wax printing. Air-gap PADs consist of paper test zones affixed to a hydrophobic backing; the spaces between test zones provide an “air gap” that the liquid cannot cross. Similar devices made of paper affixed to a hydrophobic backing have been characterized previously, but the possibility of mass-producing air-gap devices had yet to be explored.

The 12-lane PAD was developed to screen for substandard and falsified pharmaceuticals in low- and middle-income countries and has since been adapted to screen chemotherapy agents (see for example, Smith et al., J Glob Oncol, 2018, 1-10; Eberle et al., J Glob Oncol, 2020, 6, 407-413) and illicit drugs (Lockwood et al., J Forensic Sci, 2020, 65, 1289-1297). The paper titrator was developed to enable hands-on, inquiry-based analytical chemistry labs for distance learning. Each 12-lane PAD is individually serialized (starting at 10,000), and as of the time of this writing, the serial numbers had just crossed the 70,000 mark, which means that ˜7500 12-lane PADs have been produced each year since 2014; we have also produced over 34,000 titrators since 2020. For both of these devices, then, scalability was a top priority. Thus, while the air-gap PAD can be made by hand for prototyping purposes, it is designed to be compatible with large-scale roll-to-roll manufacturing.

In this study, we investigate design considerations (dimensions, wetting behavior, reagent compatibility) of the air-gap PAD and compare the performance of wax-printed and air-gap versions of 12-lane PADs and paper titrators. We also report on a pilot-scale roll-to-roll production run of air-gap PADs.

Roll-to-roll manufacturing offers continuous, in-line processing for large-scale production of microfluidic devices. Roll-to-roll manufacturing has been used to produce lateral flow assays, aquarium test strips, urine test strips, and pH strips for years, but few researchers have applied this technology to μPAD fabrication.

Accordingly, the present disclosure provides for multilane testing PADs, and methods of fabricating and using the same. In one embodiment, a paper analytical device (PAD) comprises one or more laminated strips comprising a porous hydrophilic substrate and a first adhesive layer, wherein the one or more laminated strips are disposed on hydrophobic backing with an air gap barrier between each of the one or more laminated strips disposed on the hydrophobic backing, wherein the air gap barrier is about 1 mm to about 5 mm in width and extends along an entire length of the one or more laminated strip; one or more assay regions disposed on the one or more laminated strips wherein the one or more assay regions comprises one or more reagents for detecting an analyte; and one or more sample deposition areas on the one or more laminated strips, wherein the PAD is activated when one or more solvents travel through both the one or more sample deposition areas and the one or more assay regions due to capillary action.

The disclosure also provides for methods for fabricating a paper analytic device (PAD) comprising: i) laminating a porous hydrophilic substrate onto a first double-sided adhesive layer to provide a plurality of laminated sheets; ii) cutting the plurality of laminated sheets lengthwise to provide cut ribbons of laminated strips; iii) laminating one side of the laminated strips with a hydrophobic backing to form hydrophobic-backed strips; wherein each of the laminated strips is positioned on the hydrophobic backing such that each cut ribbon is adjacent to at least one other cut ribbon with an air gap therebetween, wherein the airgap is about 1 mm to about 5 mm; and iv) recutting the hydrophobic-backed strips into test cards of a predefined size. Such methods may further comprise the steps of v) applying one or more assay reagent to predefined assay regions of each of the laminated strips; vi) attaching or printing at least one fiducial marker to each of the test cards; and vii) forming a sample deposition area on each of the laminated strips, wherein the sample deposition area is formed using a solid dosing device or liquid application strip.

In some embodiments, the methods comprise a roll-to-roll process, wherein step i) further comprises one or more of providing a continuous first roll and a continuous second roll, and step iii) further comprises a continuous third roll; wherein the continuous first roll comprises the porous hydrophilic substrate, the continuous second roll comprises the first double-sided adhesive layer, and the third continuous roll comprises the hydrophobic backing.

The disclosure also provides for methods for detecting an analyte in a sample comprising the steps of: depositing one or more samples comprising an analyte in the one or more sample deposition areas of the porous hydrophilic substrate of a PAD as described herein, wherein at least one of the one or more samples comprises a solid powder applied directly to at least one of the one or more sample deposition areas; activating the PAD by contact with a solvent to permit the sample to flow by capillary action through the one or more assay regions containing the one or more reagents for detecting the analyte to the deposition area so that the one or more assay reagents for detecting the analyte and the sample come into contact to provide visual information for analysis; and analyzing the visual information to detect the presence or the absence of the analyte in the sample.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Illustrates an embodiment of an air-gap PAD of the present disclosure.

FIG. 2. Illustrates an edge-on view of an embodiment of an air-gap PAD.

FIG. 3. Illustrates an exemplary air-gap PAD.

FIG. 4. Illustrates the application of a sample to the sample deposition area of an embodiment of the disclosure.

FIG. 5. A flowchart illustrating an embodiment of a method of fabricating an air-gap pad device.

FIG. 6. Air-gap PAD fabrication. Chromatography paper is backed with double-sided adhesive, cut into strips, applied to hydrophobic backing with air gaps between each paper strip, and sliced crossways into air-gap devices.

FIG. 7. Air-gap PAD fabrication. Simplified schematic of the roll-to-roll manufacturing process.

FIG. 8. Washburn flow plots of air-gap (transparency film, hydrophobic Trycite polystyrene, Trycite laminated with packing tape, roll-to-roll, and roll-to-roll laminated with packing tape) and wax-printed devices with 5 mm lanes. Error bars show standard deviation of 12 measurements.

FIG. 9. Air-gap (top) and wax-printed (bottom) titrators. Each square of the devices was loaded with 5 μL of the specified concentration of p-toluenesulfonic acid. 40 μL of 0.094 M sodium hydroxide with phenolphthalein was added to each square and mixed with a pipette to re-dissolve the stored TsOH. All titrators gave an endpoint (first clear, non-pink bubble) at the 800 mM TsOH square (the theoretical equivalence point was between the 700 mm and the 800 mM TsOH squares). Bottom: Titration curves obtained from ImageJ analysis of wax-printed and air-gap titrators. The endpoint occurs where the graph levels off, not at the inflection point. Error bars show standard deviations of 6 replicates.

FIG. 10. Wax-printed and air-gap pharmaceutical screening PADs. Left panels show wax-printed 12-lane PADs for ciprofloxacin (top) and isoniazid (bottom) with expected color changes circled. Right panels show air-gap PADs for ciprofloxacin (top) and isoniazid (bottom). Images of all 25 air-gap PADs read by the human evaluators can be found in the FIG. 15.

FIG. 11. Contact angles for different backing types. Error bars show standard deviations from nine replicate measurements.

FIG. 12. Air-gap device volume as a function of paper area. Error bars show standard deviations for six measurements.

FIG. 13. Air-gap device capacity by varying paper size and gap width. Transparency film was used as the backing. Error bars show standard deviation of six measurements.

FIG. 14. Air-gap device volume with different backing types. All measurements were taken with a 1 mm air gap. Error bars show standard deviation of six measurements.

FIG. 15. An embodiment of an air-gap 12-lane pharmaceutical-screening PAD images. Top row: amoxicillin, 2^{nd} row: ciprofloxacin, 3^{rd} row: isoniazid, 4^{th} row: pyrazinamide, bottom row: rifampicin.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and sub stituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

As used herein, “paper analytical device” (or “PAD”) refers to a composition based on a porous hydrophilic material/substrate (such as a paper) and comprises areas of air gaps which act as hydrophobic barriers to define hydrophilic assay regions. The hydrophilic assay regions may include one or more assay reagents to detect an analyte in a sample.

As used herein, the term “hydrophobic-backed strips” refers to a laminated strip (formed of a cut ribbon of porous hydrophilic substate and double-sided adhesive layer laminated to one side) that is adhered to or laminated for a hydrophobic backing material.

Generally, a “sample” refers to any composition or other material that includes at least one target analyte that a user wishes to investigate. In some embodiments, a sample may include a biosample such as, but not limited to, blood, serum, plasma, buffy coat, wound exudates, pus, lung and other respiratory aspirates, nasal aspirates, bronchial lavage fluids, saliva, sputum, medial and inner ear aspirates, cyst aspirates, cerebral spinal fluid, feces, urine, tears, mammary secretions, ovarian contents, ascites fluid, mucous, stomach fluid, gastrointestinal contents, urethral discharge, synovial fluid, peritoneal fluid, vaginal fluid or discharge, amniotic fluid, semen or the like. Assay from swabs or lavages representative of mucosal secretions and epithelia are also anticipated, for example mucosal swabs of the throat, tonsils, gingival, nasal passages, vagina, urethra, anus, and eyes, as are homogenates, lysates and digests of tissue specimens of all sorts. Besides physiological fluids, samples of water, pharmaceuticals, food products, air filtrates, and so forth may also be test specimens.

As used herein, a “camera device” refers to a device that contains a camera component. Exemplary camera devices are various types of digital cameras and scanners as well as mobile devices such as cell phones, smartphones or similar devices. When digital cameras are used, the images will be uploaded to a computer or other electronic device that is capable of transmitting the pictures to another location. Of course, when a picture or image is taken from a mobile phone, the phone already generally has the ability to forward the image, e.g., as a text message or as part of an e-mail.

### Embodiments of the Invention

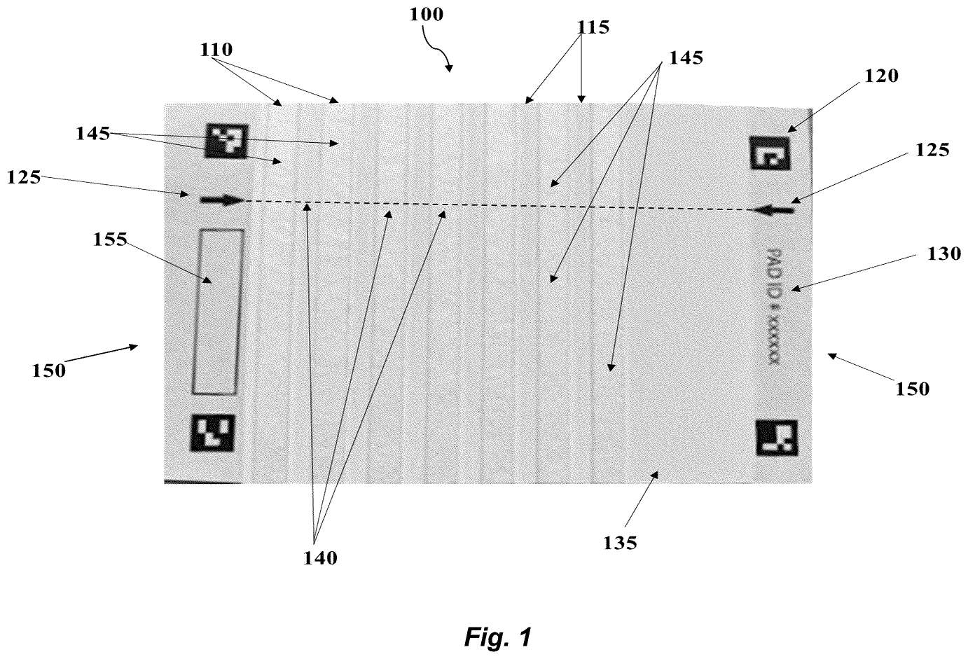

This disclosure relates to paper analytical devices (PADs) and methods for using and making the same. Generally, embodiments of PADs may comprise one or more laminated strips comprising a porous hydrophilic substrate and adhesive layer, wherein the laminated strips are positioned on hydrophobic backing. An air gap barrier is positioned between each of the laminated strips that is disposed on the hydrophobic backing, such that the air gap barrier is about 1 mm to about 5 mm in width and extends along an entire length of the laminated strip. In some embodiments, one or more assay regions are disposed on the one or more laminated strips wherein the one or more assay regions comprises one or more reagents for detecting an analyte, and one or more sample deposition areas are disposed, for example, at a swipe line about ⅓ to about ½ way down from the top of each of the laminated strips. The term “swipe line” refers to one embodiment of the PAD used for analysis of powdered, crystalline, or other solid analytes, which can be smeared, wiped, or swiped in one motion across multiple laminated strips to deposit analyte in multiple sample deposition regions; in FIG. 1, the “swipe line” is defined by the line connecting the arrows numbered 125. In some cases, assay regions and sample deposition areas may be in the same location on the laminated strip, such that the sample is deposited on top of the assay region. The PAD is activated when one or more solvents travel through both the one or more sample deposition areas and the one or more assay regions due to capillary action. The solvent may pass through the one or more assay regions before, after, or both before and after it passes through the one or more sample deposition areas.

Preferably, the laminated strips of the disclosure comprise a hydrophilic substrate and an adhesive layer. The hydrophilic substrate and the double-sided adhesive layer may be laminated together using thermal lamination or dry lamination techniques.

In some embodiments, the porous, hydrophilic substrate comprises a paper such as a fast chromatography paper or an absorbent blotting paper. A suitable porous hydrophilic substrate includes one that has a fast flow rate via capillary action and enough absorption capacity to hold adequate amounts of reagent. Lastly, it is preferable that the hydrophilic substrate is compatible with at least one of the methods used to fabricate the one or more assay regions of the device. Characteristics which should be considered when designing an analytical device of the invention, such as a PAD, include solvent flow-rate through the unfabricated substrate, printability, density, thickness, pH, basis weight, solvent flow-rate through fabricated substrate, compatibility with fabrication methods, pore size, and porosity. Preferred characteristics include high solvent flow-rate through unfabricated substrate and a comparable solvent flow-rate through fabricated substrate, a substrate that is flexible, but sturdy enough to resist tearing when a solid sample is wiped or spread across the sample deposition area, a thickness around about 0.1-0.5 mm, pH relatively close to neutral, resistance to deterioration caused by fabrication methods and materials, and a large pore size. Examples of suitable materials for a PAD, include, but are not limited to nitrocellulose acetate, chromatography paper, cellulose acetate, cellulosic paper, filter paper, tissue paper, writing paper, paper towel, cloth, and porous polymer film. In some embodiments, the hydrophilic substrate for a PAD is Whatman 3 mM CHR chromatography paper, Ahlstrom 205, Ahlstrom 222, Ahlstrom 226, Ahlstrom 319, Whatman 1 CHR paper, or Whatman No. 1 filter paper. In some embodiments, the hydrophilic substrate is Ahlstrom 319 filter paper.

In some embodiments, the basis weight (g/m²) of the PAD can range from about 50 g/m² to about 400 g/m². Specific preferable basis weights include about 90, 176, 187, 192, and 307 g/m². In some embodiments, the thickness of the PAD may be about 0.10 mm to about 1 mm, or about 0.4 mm to about 0.85 mm. In some embodiments, the PAD can have a pH ranging from about 5.5 to about 8.0. In some embodiments, a pore size may range from about 10.0 micrometers to about 30 micrometers.

In some embodiments, the adhesive layer is a double-sided adhesive layer. In some embodiments, the double-sided adhesive layer is a layer of double-sided tape. In other embodiments, the adhesive layer is an adhesive that is suitable for thermal lamination or dry lamination (e.g., a vinyl adhesive, a sheet sprayed on one or both sides with an adhesive, etc.).

Embodiments of the PADs also may include a hydrophobic backing that is typically made of water-insoluble, non-porous and rigid material and has a length and width equal to or greater than the layers situated thereon. In preparation of the backing, various natural and synthetic organic and inorganic materials can be used, provided that the backing prepared from the material should not hinder capillary actions of the absorption material, nor non-specifically bind to an analyte, nor interfere with the reaction of the analyte with a detector. Representative examples of polymers usable in the present invention include, but are not limited to, polyethylene, polyester, polypropylene, poly(4-methylbutene), polystyrene, polymethacrylate, poly(ethylene terephthalate), nylon, poly(vinyl butyrate), glass, ceramic, metal, and the like. In some embodiments, the hydrophobic backing comprises polystyrene or hydrophobically treated polystyrene such as Trycite®.

Embodiments of a PAD also include one or more assay regions that may comprise one or more assay reagents deposited on the surface of the hydrophilic substrate of each laminated strip. Typically, a laminated strip will contain about 1 assay region, about 2 assay regions, about 3 assay regions, about 4 assay regions, about 5 assay regions, about 6 assay regions, about 7 assay regions, about 8 assay regions, about 9 assay regions, or about 10 assay regions. In some embodiments, a laminated strip comprises greater than 10 assay regions. Each assay region may include one or more assay reagents or precursors thereof capable of identifying an analyte in a sample applied to the hydrophilic substrate of the laminated strip. Samples may be applied as solids (e.g., powders) or as solutions.

The various reagents or reagent forming precursors can be loaded into the assay regions directly before use or stored in the assay regions in dry form. The reagents or precursors can be loaded into the reaction area individually by hand, or via an automated process. Generally, the regents are loaded as liquid solutions or suspensions, and allowed to dry prior to use of the PAD. Examples of reagent materials suitable for use in the PAD include, but are not limited to, Folin-Ciocalteu, potassium hexacyanoferrate(II) trihydrate, iodine-potassium iodide reagent, universal indicator, ferric chloride, triiodide, triiodide-starch complex, soluble starch; cationic, anionic, and neutral pH indicators; barium chloride, sodium rhodizonate, potassium hexacyanoferrate(II), NaOH, tosic acid, potassium carbonate, citric acid, copper sulfate, sodium tetraphenylborate, cobalt thiocyanate, ammonium molybdate, nitroaniline, 1,2-napthaquinone-4-sulfonate, dimethylglyoxime, and paradimethylaminobenzaldehyde. These reagents may be deposited from aqueous solution or from organic solution. The upper limit on the amounts of reagents added is more or less determined by the PAD's loading capacity. The volume of the reagent loaded onto the PAD can range from about 1 microliter to about 100 microliters, or from about 2 microliters to about 10 microliters, or preferably from about 2 microliters to about 4 microliters.

Reagents may be deposited on the surface of the analytical device in many ways that will be familiar to those skilled in the art, including but not limited to the use of: microcapillary pipettes and droppers, single- or multi-channel automatic pipetting devices, rods that can capture a droplet of solution or 96-spoke inoculators that perform this function with multiple rods simultaneously, dipping or spraying equipment, or solution deposition robots. In one embodiment, the reagents are manually deposited using an automatic pipette. In another embodiment, reagents are deposited using a 96-spoke inoculator. In another embodiment, the reagents are deposited using a Biomek 96-well-plate replicating robot that can deposit 8, 12, or 96 reagent spots simultaneously, or a Sequence Biotech Plus 96-wellplate pipettor. (see, for example, Bliese et al., Anal. Methods, 2019, 11, 4727-4732).

In some embodiments, one or more laminated strips may be arranged parallel to one another on the hydrophobic backing such that an air gap is present between each of the parallel hydrophobic strips and extends along the entire with of the laminated strip to form a hydrophobic barrier between said laminated strips. In some embodiments, about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 laminated strips are positioned on hydrophobic backing. Typically, laminated strips are arranged in parallel patterns of about 4 to about 15 on the hydrophobic backing so that they can be easily cut into test cards or test strips of predetermined size, thereby producing a testing device. In some embodiments the testing device may be adhered to a card holder or other substrate to support the device. In some embodiments, 6, 7, 8 12, 13, or 14 laminated strips are positioned onto the hydrophobic backing to form a testing device. In some embodiments, the laminated strips are laminated to the hydrophobic backing.

In some embodiments, the laminated strips are about 2 mm to about 15 mm in width or about 2 mm to about 7 mm in width. In some embodiments, the air gap barrier is about 1 mm in width, about 2 mm in width, about 3 mm in width, about 4 mm in width, about 5 mm in width, about 6 mm in width, about 7 mm in width, about 8 mm in width, about 9 mm in width, or about 10 mm in width. In some embodiments, the air gap barrier is about 2 mm to about 4.5 mm in width.

Exemplary PADs contain at least one electronically readable information zone which provides information necessary for determining the outcome of the test performed on the PAD based on images obtained by a camera device. The information zone typically includes appropriate information that is electrically readable per se or after being photographed, or otherwise imaged electronically. Such information may include an identification tag such as a two-dimensional bar code (e.g., a QR or ArUco code), color standards, and/or fiducial or alignment marks.

Each PAD can be imprinted with a two-dimensional barcode such as a Quick Response (QR) barcode that contains the type and serial number of the PAD so that a PAD test can be uniquely identified and the necessary color processing steps to perform the test can be automatically determined, which provides a simple and inexpensive way to uniquely identify the PAD, in addition to providing pertinent information for perspective distortion correction and subsequent color analysis. Depending on the application, other information can also be encoded in the two-dimensional code image. A key task of image analysis software is the perspective correction or transformation of distorted images, which transforms an image captured at an unknown standoff and optical axis position to a canonical coordinate system in which regions to be analyzed for subsequent color characterization are expressed. The origin and basis vectors for this coordinate system can be automatically calculated from the position of “finder marks” or fiducial marks on the QR or ArUco code. In some embodiments, each PAD may also contain one or more additional fiducial markers such as “finder squares” or rectifiers to eliminate angle and 3D distortions of the PAD's photographed image. The identification zone can be placed anywhere on the PAD. Preferably, the fiducial marks or alignment marks can be printed on the PAD (e.g., on the hydrophobic backing) prior to application of the laminated strips and the identification tag is located on one side of the PAD (e.g., the right side). Exemplary identification zones and uses thereof are described, for example in U.S. Pat. No. 9,354,181 to Barstis et al.

Optionally, some embodiments of a PAD may include a color calibration using the color calibration zone on the PAD, which consists of different colored sub-regions, including a white region and a black region. Image analysis software can be used to compare the extracted colors in the PAD image's color calibration zone to known values to identify the specific color correction methods needed. One such method is white balancing, in which the overall brightness of the image is adjusted to force the white square in the PAD image to have a pure white color value. The calibration zone can be in any suitable shape, including rectangles, squares, circles, or triangles. The sub-regions can be in any suitable shape, including rectangles, squares, circles or triangles. Preferably, the color calibration zone is a rectangle region and the sub-regions are different colored squares. The color calibration zone can be printed onto the hydrophobic backing card prior to or after application of the laminated strips, or it can be placed directly on the laminated strips. The calibration zone can be placed anywhere on the PAD. Preferably, the calibration zone is printed on the backing card prior to application of laminated strips and is located on an upper corner of the PAD.

Thus, in some embodiments, a PAD may comprise, for example, an information zone having multiple fiducial marks and another information zone comprising a QR or ArUco code or other identification tag. Optionally, the PAD also may comprise a color calibration zone.

FIG. 1 depicts an exemplary PAD. PAD 100 includes laminated strips 110 and air gap barriers 115 forming hydrophobic barriers between each laminated strip 110. One or more assay regions 145 are distributed along the length of each laminated strip 110. The number of assay regions on each laminated strip 110 may vary based on the target analyte. Sample deposition areas 140 may be located in line with swipe line 125 (the two arrows 125 define the swipe line (dotted line)). Preferably, the sample deposition area 140 is located about ⅓ to about ½ way down the from the top edge of the laminated strip 110. It is contemplated that a sample deposition area may located at any position along the length of the laminated strip (e.g., the middle of the strip or at either end). Each laminated strip also may have the sample deposition area located in a different position or the same position as an adjacent laminated strip. A PAD also may include one or more electronically readable information zones 150. The electronically readable information zones 150 may include information for identifying, orienting, and cataloging PADs and their images. For example, electronically readable information zones 150 may include one or more fiducial markers 120 for orienting an image and facilitating analysis of an image of the PAD, a unique PAD identifier number 130, and a space for written or printed notes 155. The electronically readable information zones 150 also may include one or more QR codes. In some embodiments, the one or more electronically readable information zones 150 also may include assay regions 145 and sample deposition area 140. In some embodiments, the electronically readable information zones 150 include some or all laminated strips 110.

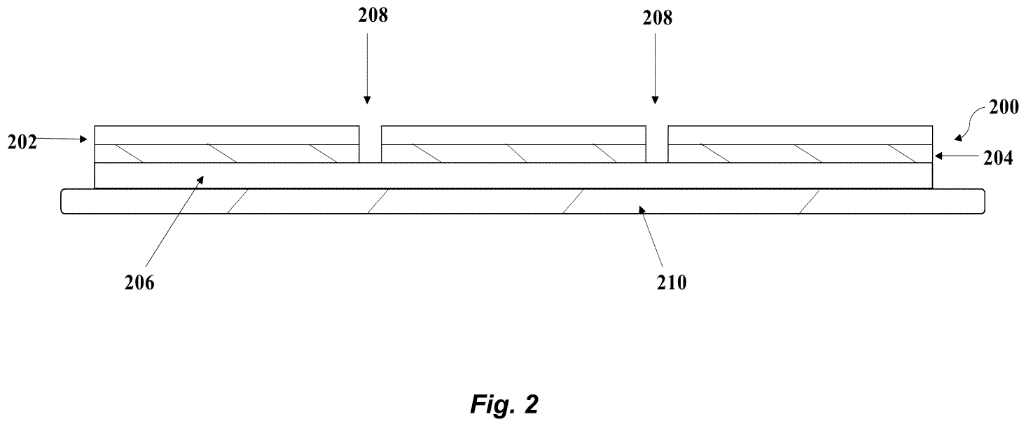

FIG. 2 depicts an edge-on view of a PAD. Laminated strip 200 includes a hydrophilic substrate layer and a double-sided adhesive layer 204. The laminated strip 200 is adhered to hydrophobic backing 206, preferably via lamination. Optionally, the laminated strip and the hydrophobic backing may be adhered to a card holder 210, which may improve the mechanical strength of the PAD and/or serve as a substrate for printing of the electronically readable information zones. As can be seen, air gaps 208 form a hydrophobic barrier between the laminated strips 200. The air gap barriers 208 are defined by the distance between the laminated strips 200 and the hydrophobic or solvophobic nature of the hydrophobic backing 206.

In some embodiments, a camera device may be used for capturing an image of the PAD that has reacted with the composition to be analyzed. In some embodiments, the image may be captured using the camera device and providing an image analysis software capable of using information provided by the information zone and the image of the test result in order to identify and quantify a colorimetric change within the assay region of the PAD shown in the captured image. In some embodiments, the captured image contains a two-dimensional bar code such as a QR or ArUco code and one or more fiducial markers. The image software identifies the QR code region, separates the image of the PAD's assay regions from background present in the picture, scales, rotates, and performs geometrical transformations on the captured PAD image based on the QR code and the one or more fiducial markers, aligns the PAD assay regions with stored images in the database, reads test results from pre-specified locations in the stored assay regions, and classifies the test results. The method of the invention further comprises compiling a database of the captured images of the paper analytical devices and the computed test outcomes, wherein the two-dimensional barcode is a QR or ArUco code that allows for automated identification of a specific PAD including such information as the PAD-type, serial number and/or fabrication date.

In some embodiments, image analysis software is provided on the camera device for processing the captured image in situ. Alternatively, the image analysis software may be provided on a network server such that the captured image is processed by sending the picture to the network server that performs the analysis and transmits the results back to the camera device.

The analytes to be detected can be in any suitable formulation, including tablets, pills, solids, or powders. Other suitable formulations include liquids, such as suspensions, syrups, or solutions of medications. In some instances, a solid formulation for investigation can be used directly with the PAD, by swiping, rubbing, pressing, or crushing the formulation onto the PAD at a specific location(s)(e.g., sample deposition areas). In other instances, a solid formulation must be diluted into a liquid solution or suspension in order to be used with the specific PAD. Liquid formulations may be added directly to the PAD or may be further diluted and then added to the PAD. In some instances, a formulation may be used both directly, and also as a dilution on the same PAD. Preferably, the sample is applied to a sample deposition area on one or more laminated strip. It is contemplated that a sample deposition area may be positioned at any location along the length of the laminated strip, for example, in the middle of the laminated strip, and more preferably, at about ⅓ to about ½ of the way down from the top edge of the laminated strip.

Various analytes include classes of treating agents such as anti-malarials (artemether, lumifantrine), beta lactam antibiotics (ampicillin, amoxicillin), cox-inhibitors, anti-parasitic drugs (albendazole, mebendazole, ivermectin), antipyretics (aspirin, acetaminophen) phosphodiesterase inhibitors (sildenafil citrate), and anti-virals (ostamilvir phosphate). They can also be used to analyze foodstuffs that have been supplemented or fortified with micronutrients (iodine, iron, zinc, vitamin C) or with medications (diethylcarbamazine citrate). However, other classes of active agents are also contemplated, such as NSAIDs (ibuprofen), analgesics (lidocine), HMG-CoA reductase inhibitors (statins), ace-inhibitors (quinapril), macrolide antibiotics (erythromycin), anti-anxiety medications (alprazolam), bi-polar disorder and schizophrenia medications (olanzapine), anemia medications (epoetin alfa), and anti-retrovirals (abacavir). In some embodiments, the various analytes may comprise one or more of albendazole, amoxicillin, ampicillin, azithromycin, benzylpenicillin, ceftriaxone, chloroquine, ciprofloxacin, doxycycline, epinephrine, ethambutol, ferrous sulfate, hydroxychloroquine, isoniazid, promethazine hydrochloride, pyrazinamide, rifampicin, sulfamethoxazole, tetracycline, and a mixture of rifampicin/isoniazid/pyrazinamide/ethambutol (RIPE).

In other embodiments, the analyte is a chemotherapeutic agent. Exemplary chemotherapeutic agents include, but are not limited to, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-fu), 6-mercaptopurine (6-mp), capecitabine (xeloda), cladribine, clofarabine, cytarabine (ara-c), decitabine, floxuridine, fludarabine, gemcitabine (gemzar), hydroxyurea, methotrexate, nelarabine, pemetrexed (alimta), pentostatin, pralatrexate, thioguanine, trifluridine/tipiracil combination, daunorubicin, doxorubicin (adriamycin), doxorubicin liposomal, epirubicin, idarubicin, valrubicin, bleomycin, dactinomycin, mitomycin-c, mitoxantrone, irinotecan, irinotecan liposomal, topotecan, etoposide (vp-16), mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, all-trans-retinoic acid, arsenic trioxide, asparaginase, eribulin, hydroxyurea, ixabepilone, mitotane, omacetaxine, pegaspargase, procarbazine, romidepsin, and vorinostat. In some embodiments, the chemotherapeutic agent is one or more of cisplatin, oxaliplatin, doxorubicin, and methotrexate. In some embodiments, the chemotherapeutic agent is one or more of cisplatin, carboplatin, oxaliplatin, methotrexate, doxorubicin, and sodium 2-mercaptoethane sulfonate (MESNA).

In other embodiments, the analyte is an illicit drug. Exemplary illicit drugs (including drugs of abuse or controlled substances) that maybe be detected using the subject invention include, but are not limited to: amphetamine, methamphetamine, 3,4-methyl enedi oxymethamphetamine (MDMA a.k.a., Ecstasy), barbiturates (such as amobarbital, butalbital, pentobarbital, phenobarbital, and secobarbital), benzodiazepines (such as alprazolam and diazepam); cannabinoids (such as hashish and marijuana), cocaine, fentanyl, LSD, methaqualone, opiates (such as heroin, morphine, codeine, hydromorphone, hydrocodone, methadone, oxycodone, oxymorphone, and opium), phencyclidine, and propoxyphene. In certain embodiments detection of prescription drugs, which are commonly abused, such as pain killers (oxycodone, percocet, etc.) may be detected as well as prescription drugs not commonly subject to abuse. In some embodiments, the illicit drug comprises one or more of cocaine, cocaine HCl, methamphetamine, heroin, and (MDMA).

In some embodiments, the target analyte is found in a test sample such as blood, serum, plasma, buffy coat, wound exudates, pus, lung and other respiratory aspirates, nasal aspirates, bronchial lavage fluids, saliva, sputum, medial and inner ear aspirates, cyst aspirates, cerebral spinal fluid, feces, urine, tears, mammary secretions, ovarian contents, ascites fluid, mucous, stomach fluid, gastrointestinal contents, urethral discharge, synovial fluid, peritoneal fluid, vaginal fluid or discharge, amniotic fluid, semen or the like. In other embodiments, the test sample comprises the analyte is a solid sample, such as a powder substance or a powder derived from a crushed tablet or the like, or a solid that is otherwise insoluble or poorly soluble in a solvent.

Thus, in some embodiments, a PAD comprises one or more laminated strips comprising a porous hydrophilic substrate and a first adhesive layer, wherein the one or more laminated strips are disposed on hydrophobic backing with an air gap barrier between each of the one or more laminated strips disposed on the hydrophobic backing, wherein the air gap barrier is about 1 mm to about 5 mm in width and extends along an entire length of the one or more laminated strip; one or more assay regions disposed on the one or more laminated strips wherein the one or more assay regions comprises one or more reagents for detecting an analyte; and one or more sample deposition areas on each of the one or more laminated strips, wherein the PAD is activated when one or more solvents travel through both the one or more sample deposition areas and the one or more assay regions due to capillary action.

In some embodiments, the PAD further comprises a second adhesive layer laminated to the hydrophobic backing. In some embodiments, the PAD is disposed on a backing card of a defined width and length.

In some embodiments, a PAD comprises at least one optically readable information area that provides visual information for detection of analyte, wherein the at least one optically readable information area comprises one or more alignment references for transforming or correcting a captured image of the PAD to facilitate analysis. In some embodiments, the one or more alignment references include a plurality of fiducial markers for orienting a captured image of the PAD, and the PAD further comprises an identification tag of a two-dimensional barcode. In some embodiments, the visual information comprises color information caused by an interaction of the analyte and the one or more reagents.

In some embodiments, the width of a laminated strip is about 2 mm to about 7 mm and the width of the air gap barrier is about 2 mm to about 4.5 mm in width.

In some preferred embodiments, the analyte comprises one or more of an antimicrobial agent, a chemotherapeutic agent, or an illicit drug. In some embodiments, the antimicrobial agent comprises one or more of albendazole, amoxicillin, ampicillin, azithromycin, benzylpenicillin, ceftriaxone, chloroquine, ciprofloxacin, doxycycline, epinephrine, ethambutol, ferrous sulfate, hydroxychloroquine, isoniazid, promethazine hydrochloride, pyrazinamide, rifampicin, sulfamethoxazole, tetracycline, and a mixture thereof. In some embodiments the chemotherapeutic agent comprises one or more of cisplatin, oxaliplatin, methotrexate, sodium 2-mercaptoethane sulfonate (MESNA) and doxycycline. In some embodiments, the illicit drug comprises one or more of an opiate or synthetic opioid, cocaine, 3,4-methylenedioxymethamphetamine (MDMA), and methamphetamine.

In some embodiments, the sample comprising the analyte comprises a solid powder applied directly to the one or more sample deposition areas.

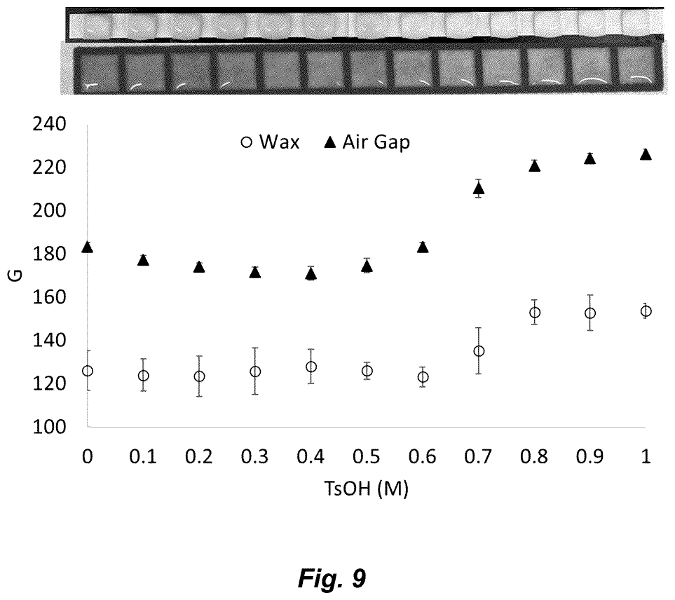

In some embodiments, the air gaps or other hydrophobic regions may also define control regions within the hydrophilic substrate. For example, a timer region may be included in order to alert the user when the test has completed. The timer region may comprise a colorimetric indicator. Additionally, the PAD may include positive or negative control regions. A negative control may be included in order to verify the purity of the reaction solvent. A positive control may be included in order to verify the presence (or absence) of the chemical to be detected. The control substances, if any, may be included in the paper medium at the time the other colorimetric reagents are added to the paper medium. The PAD may also contain hydrophilic regions for titrations and/or reverse titrations (see FIG. 9), as well as user compliance lanes for improving the accuracy of the quantitative analysis of the target analyte.

In some embodiments, a paper titrator may comprise a plurality of laminated strips shaped into squares (usually about 6, 7, 12, 13, or 14) and affixed to a hydrophobic backing and separated by air gap barriers. Usually, the laminated squares will be about 5 mm square, and the air gap barriers will be about 4 mm. Each laminated square may be pre-loaded with a known amount of reagent (usually a solution of an acid such as p-toluene sulfonic acid, potassium hydrogen phthalate, or another suitable strong acid, or a strong base such as sodium hydroxide or potassium hydroxide, or a redox-active reagent such as triiodide stabilized with povidone). Each laminated square may be loaded with a different concentration reagent. A solution of an analyte (a base such as sodium hydroxide or another base, an acid such as acetic acid, potassium hydrogen phthalate, hydrochloric acid, or another acid, an amphiprotic substance such as a tartrate salt or sodium bicarbonate, or a redox-active substance such as ascorbic acid) may be added to each laminated square along with a suitable indicator (such as phenolphthalein or universal indicator for acid-base titrations, or starch for a iodometric titration). When the amounts of reagent and analyte are equal, the indicator produces a color change. The analyte concentration can then be calculated from the amount of reagent in the square where the color change occurs.

For example, in some embodiments, a 13-square titrator might have squares loaded with 5 microliters of 0, about 0.1 M, about 0.2 M, about 0.3 M, about 0.4 M, about 0.5 M, about 0.6 M, about 0.7 M, about 0.8 M, about 0.9 M, about 1.0 M, about 1.5 M, and about 3.0 M a base such as sodium hydroxide. This titrator could then be used, for example, to determine the concentration of an acid such as acetic acid or an amphiprotic compound such as potassium bitartrate using an acid-base indicator such as phenolphthalein. In other embodiments, a 13-square titrator might be loaded with 5 microliters of 0, about 0.1 M, about 0.2 M, about 0.3 M, about 0.4 M, about 0.5 M, about 0.6 M, about 0.7 M, about 0.8 M, about 0.9 M, about 1.0 M, about 1.5 M, and about 3.0 M of an acid such as p-toluene sulfonic acid, and used to determine the concentration of a base such as sodium hydroxide or an amphiprotic compound such as sodium bicarbonate using an acid-base indicator such as phenolphthalein. In still other embodiments, a titrator might be loaded with about 10 microliters per square of povidone-iodine solutions ranging from about 5 micrograms per microliter to about 50 micrograms per microliter and used to determine the ascorbic acid concentration of a vitamin C solution or a fruit juice such as apple juice using a starch indicator.

Embodiment of the disclosure also provides methods for detecting the presence or absence of an analyte in a sample composition, or for quantifying the amount of at least one analyte in a sample composition, or for comparing the amounts of two or more analytes present in a sample composition. Generally speaking, once the sample has been applied to the one or more deposition areas, the movement of a solvent, by means of capillary action or by manual or automated deposition, into the sample deposition areas and the assay regions typically causes a colorimetric change in each region that can be analyzed to detect the presence/absence of the analyte in the composition, to quantify the amount of the targeted analyte, or to compare the amounts of two or more analytes present in the sample.

In some embodiments, a method comprises providing a paper analytical device of the disclosure; depositing one or more samples comprising an analyte in the one or more sample deposition areas of the porous hydrophilic substrate of the PAD, wherein at least one of the one or more samples comprises a solid powder applied directly to at least one of the one or more sample deposition areas; activating the PAD by contact with a solvent to permit the sample to flow by capillary action from the deposition area to the one or more assay regions containing the one or more reagents for detecting the analyte so that the one or more assay reagents for detecting the analyte and the sample come into contact to provide visual information for analysis; and analyzing the visual information to detect the presence or the absence of the analyte in the sample. Alternatively, all or some of the samples may be of a liquid or solution form.

In some embodiments, providing the visual information for analysis comprises causing a color change that is analyzed to detect the presence or the absence of the analyte in the sample when the one or more assay reagents for detecting the analyte and the sample come into contact. Generally speaking, the one or more assay reagents detect a certain functional group of the target analyte. Thus, PADs use multiple test strips (e.g., 6 or 12) lanes, each with one or more reagents configured to detect a certain functional group to produce a color fingerprint of the target analyte. Due to the complex interactions of the functional groups within molecules, the color test in a lane need not give a simple presence/absence result; for many reagents that will be known to one skilled in the art, molecules that have the same functional group may produce different colors in the assay region. The color fingerprint of each test PAD can be compared to a database of digital images of control PADS that are positive or negative for a given analyte to determine, for example, the presence or absence of the analyte and the amount of the analyte present in the sample. Examples of assay reagents and color fingerprints that identify each target analytes are disclosed, for example, in Bliese et al., Anal. Methods, 2019,11, 4727-4732; Lockwood et al., J Forensic Sci, July 2020, Vol. 65, No. 4; and Smith et al., J Glob Oncol. 2018 Dec.; 4:1-10.

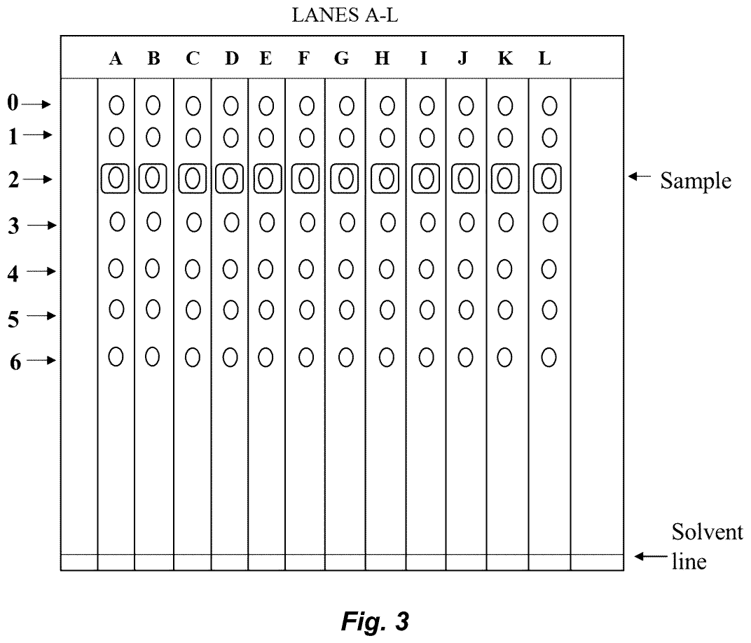

FIG. 3 illustrates an exemplary test area of a 12-lane PAD with an embodiment of an assay reagent layout. The 12 lanes (i.e., laminated strips) are labeled A-L. The sample deposition area is indicated by the sample arrow. Assay regions with assay reagents can include any of positions 0-6 of each lane. In some embodiments, the sample deposition area may overlap an assay region. Each lane detects one or more chemical groups and contains a specific set of reagents such as is listed in Table 1 where the row refers to a position of 0-6. For example, lane A, a timer lane, contains reagents at position 0 and 3.

TABLE 1
Sample reagents in 12-lane PAD.

| Test | Lane | Row | Reagent |
|---|---|---|---|
| Timer | A | 0 | NiCl₂ 0.20M |
| | | 3 | Dimethyl glyoxime DMG 0.20M |
| Ninhydrin | B | 2 | Ninhydrin 30 mg/ml |
| | | 3 | Ninhydrin 30 mg/mL |
| | | 4 | K₂CO₃ 2M |
| | | 5 | K₂CO₃ 2M |
| Biuret Reagent | C | 3 | Biuret Reagent |
| | | 4 | Biuret Reagent |
| Acidic cobalt | D | 3 | [Co(SCN)₄]²⁻ 1M |
| thiocyanate | | 4 | [Co(SCN)₄]²⁻ 1M |
| | | 5 | Tosic acid (1M HOTs) |
| | | 6 | Tosic acid (1M HOTs) |
| pH 8 cobalt | E | 3 | [Co(SCN)₄]²⁻ 1M |
| thiocyanate | | 4 | [Co(SCN)₄]²⁻ 1M |
| | | 5 | Tris Buffer pH 8 1M |
| | | 6 | Tris Buffer pH 8 1M |
| Beta Lactum | F | 1 | CuSO₄*5H₂O 1M |
| | | 2 | K₂CO₃ 2M |
| Plain SNP | G | 1 | NaOH 4M |
| | | 2 | NaOH 2M |
| | | 4 | Sodium Nitroprusside |
| | | | 100 mg/mL (orange color) |
| | | 5 | Sodium Nitroprusside |
| | | | 100 mg/mL |
| NQS | H | 1 | NaOH 4M |
| | | 3 | NQS 10 mg/mL |
| | | 4 | NQS 10 mg/mL |
| Ethambutol | I | 1 | NaOH 2M |
| | | 3 | CuSO₄*5H₂O 1M |
| Starch | J | 3 | Triiodine and 2% Povidone |
| | | 4 | Triiodine and 2% Povidone |
| Phenols | K | 1 | NaOH 2M |
| | | 2 | NaOH 2M |
| | | 4 | Tosic acid (1M HOTs) |
| | | 5 | P-Nitroaniline 30 mg/mL |
| | | 6 | NaNO2 30 mg/mL |
| Carbonate | L | 3 | FeCl3 50 mg/mL |
| | | 4 | FeCl3 50 mg/mL |

TABLE 2
Overview of chemistry of 12-lane PAD

| Lane | Description of Chemistry |
|---|---|
| A | Timer lane. Water moving up the lane by capillary action |
| | carries a chelating agent up to meet a spot of nickel(II) at the |
| | top of the lane. A pink dot visually indicates that the water |
| | has moved all the way up the lane. |
| B | uses ninhydrin to detect primary amines. Although ninhydrin |
| | usually is heated at 100° C. to force the formation of |
| | Ruhrman's Purple, at room temperature and in water the |
| | reaction stops after the formation of a Schiff base, whose |
| | color can range from yellow (ampicillin) or orange |
| | (isoniazid) to green (amoxicillin). |
| C | uses the Biuret reagent, which forms a green or blue colored |
| | compounds when a copper (II) ion coordinates to multiple |
| | amide groups. |
| D | [Co(SCN)₄]²⁻ pairs with two protonated tertiary amines, |
| | forming an insoluble blue or green ion-pair; the two lanes are |
| | set up to do this reaction at either acidic or neutral pH, which |
| | allow some differentiation between tertiary amines. |
| | Hydrophobic secondary amines also react, but tend to give |
| | more soluble ion pairs, so the blue or green color is often |
| | washed out in the center of the lane but visible near the wax |
| | boundary. |
| E | Same as lane D, but at a different pH; comparing lanes D |
| | and E can differentiate some tertiary amines. |
| F | uses base and copper to detect the beta lactam functional |
| | group present in ceftriaxone and many other antibiotics. A |
| | dark green insoluble complex of unknown structure is |
| | formed. |
| G | contains sodium nitroprusside, which reacts with |
| | nucleophiles to give either addition to the electrophilic NO |
| | group, or substitution of this group. |
| H | detects free thiols and other nucleophiles via a substitution |
| | mechanism with sodium 1,2-naphthoquinone 4-sulfonate |
| | (Folin's reagent) |
| I | includes copper and base in locations designed to detect the |
| | TB drug ethambutol. |
| J | contains stabilized tri-iodide ion to detect starch. |
| K | typically detects phenols via a diazonium salt mechanism. |
| | Ceftriaxone lacks a phenol, but still gives a color change in |
| | this lane because the thiazole group can be diazotized under |
| | the reaction conditions. |
| L | contains iron (III) which readily coordinates to 1,3 |
| | dicarbonyl compounds such as ciprofloxacin or levofloxacin, |
| | and to polyphenols found in many natural products. |

Some embodiments include automating the visual information for analysis by capturing an image of the PAD using a camera device; and using image analysis software configured to recognize and quantify a color change within the one or more assay regions of the PAD shown in the captured image as compared to an image of a reference standard stored in a reference database.

In some embodiments, the PAD comprises a plurality of fiducial markers for orienting the captured image; and i) correcting and/or transforming the captured image, by the image analysis software, based on the fiducial marks to align the captured image with the stored image of the reference standard in the reference database; ii) reading the color change, by the image analysis software, from pre-specified regions in the corrected and/or transformed image; and iii) determining, by the image analysis software, the presence or the absence of the analyte based on the comparison of the color changes to the stored reference standard images in the reference database.

In some embodiments, the depositing of the one or more samples in the one or more sample deposition areas comprises a use of: i) an applicator strip for liquid samples; or ii) a dosing device for solid samples, wherein the dosing device comprises an overlay template comprising one or more windows of a predefined size, wherein each of the one or more windows is configured to align with the one or more sample deposition areas, and wherein the predefined size controls an amount of the one or more samples deposited in the one or more sample deposition areas. In some embodiments, an applicator strip may be positioned perpendicular to the length of the laminated strips over a sample deposition area. The application strip can then be soaked with a test sample, or the application strip can be presoaked with the test sample prior to placement on the one or more sample deposition regions, and then the device is activated to analyze the analyte in the sample.

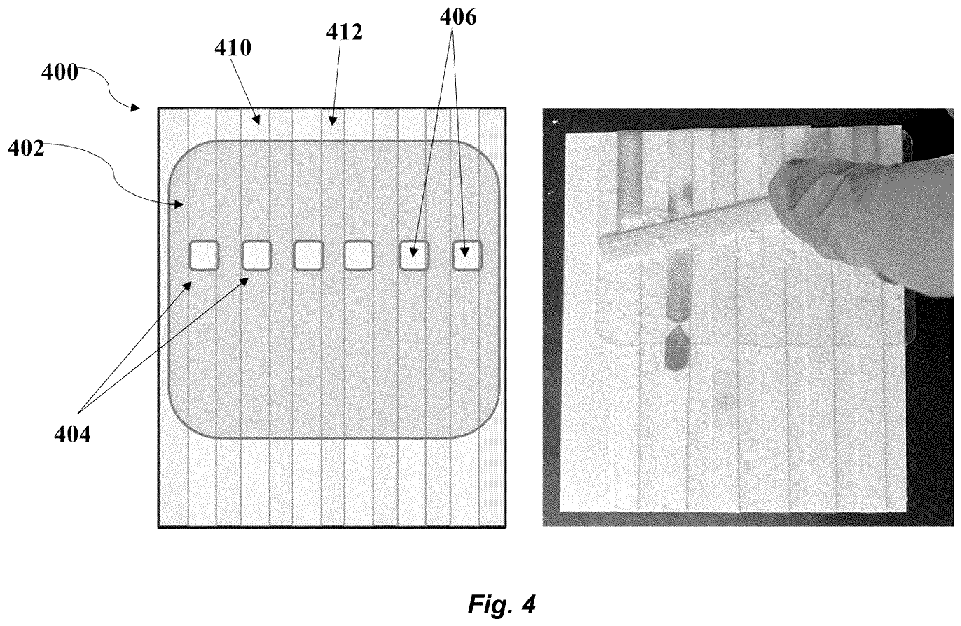

FIG. 4 shows the application of a solid sample to a sample deposition area. A dosing device 402 may be placed on and/or removably affixed to the PAD 400. The dosing device 402 includes precut windows 404 that are configured to align one or more sample deposition areas 406 on a lamented strip 410. The size and shape of windows 404 control the amount of the sample added to the PAD and prevents, along with air gap barrier 412, the mixing of said samples between laminated strips 410. In some embodiments, an applicator strip may be used instead of or in conjunction with the dosing device, to introduce the sample to the sample deposition area. Applicator strip may be presoaked in a liquid sample and positioned perpendicular across one location of the laminated strips 410. Alternatively, the applicator strip may be laid perpendicular across the laminated strips 410 or integrated into the PAD 400, and the sample may be added to the applicator strip. Generally, the applicator strip is positioned at the swipe line. Alternatively, a liquid may be deposited on the sample deposition areas using the precut window of the dosing device. In other embodiments, applicator strips 408 may be positioned on a foldable paper or plastic sheet attached to a PAD 400 such that the paper sheet is sized and shaped to align with PAD 400 and cover the laminated strips 410 when folded. In such instances, the applicator strip is positioned on the foldable sheet so that when the foldable paper or plastic sheet is folded onto the PAD, the applicator strip aligns with the swipe line and the sample deposition areas. In preferred embodiments, the swipe line corresponds to the position of the sample deposition areas (i.e., the sample deposition areas are positioned in line with the swipe line).

In some embodiments, a hydrophobic barrier, such as an air gap, can also define control regions within the hydrophilic paper medium. For example, a timer region may be included in order to alert the user when the test has completed. The timer region may comprise a color-generating reaction in which one component travels up the lane with the solvent flow and creates a color when it encounters another component at the top of the lane, or it may comprise other timing mechanisms such as delay of the solvent flow by a deposited reagent such as sugars, surfactants, or polymers. Additionally, the PAD may include positive or negative control regions. A negative control may be included in order to verify that the PAD has not become contaminated during storage or use or that the solvent used to develop the colors does not interfere with a color generating reaction. A positive control may be included to show that the reagents in a test lane are still viable, or it may be used as a standard for the image analysis software as disclosed hereinbelow. The PAD may also contain assay regions whose only function is to demonstrate that the user has complied with instructions for correct use of the PADs, or assay regions whose function is to demonstrate that the PAD is an authentic device and not a counterfeit.

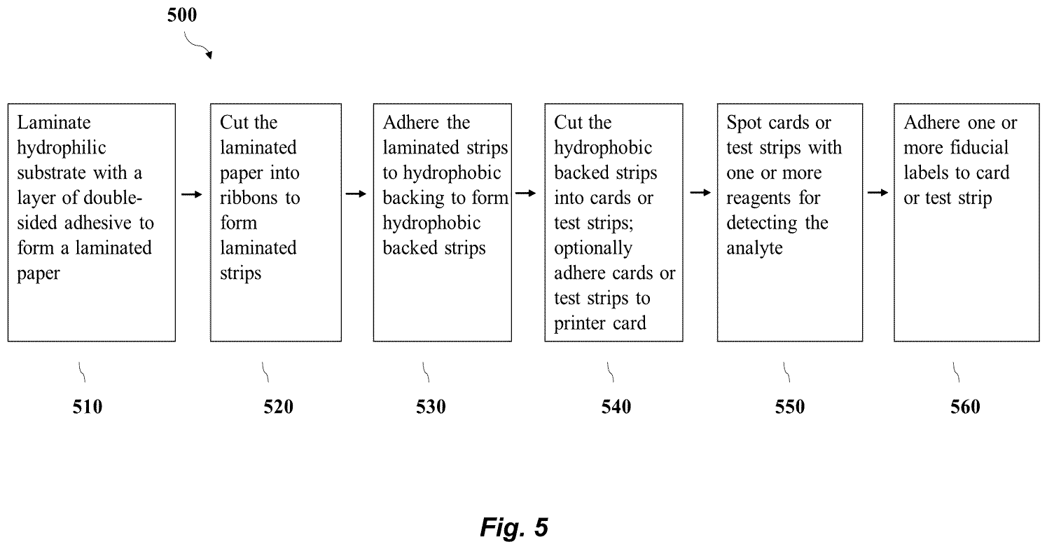

The disclosure also provides for methods of fabricating embodiments of a PAD as described herein. FIG. 5 illustrates a general method of fabricating a PAD. The method 500 comprises the steps of 510 laminating hydrophilic substrate with a layer of double-sided adhesive to form a laminated paper, 520 cutting the laminated paper into ribbons to form laminated strips, 530 adhering the laminated strips to hydrophobic backing to form hydrophobic-backed strips, 540 cutting the hydrophobic-backed strips into cards or test strips; optionally adhering the cards or test strips to custom printed card holders to support the PAD, 550 spotting the cards or test strips with one or more reagents for detecting the analyte, and 560 adhering one or more fiducial markers to card or test strip. In some embodiments, step 560 further comprises adding one or more QR or ArUco codes to the PAD.

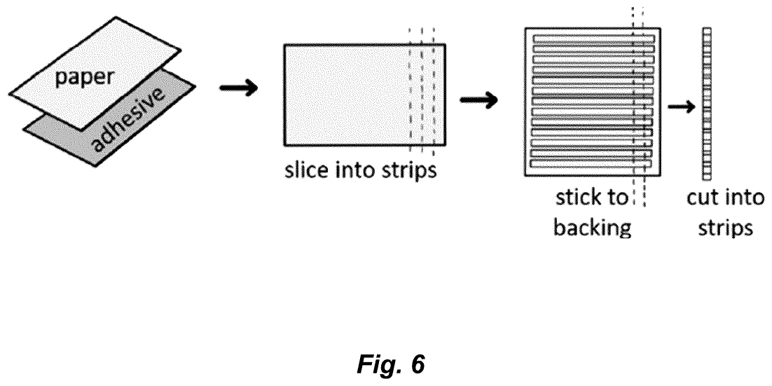

Generally, PADs are fabricated using manual methods, automated methods, or a combination of both manual and automated methods. An embodiment of a manual method of fabricating a PAD is shown in FIG. 6. This “cut and paste” method requires laminating the hydrophilic substrate to a double-sided adhesive layer using dry lamination or thermal lamination to form laminated sheets. The laminated sheets may then be cut into ribbons of desired width and lengths to form laminated strips, which are then adhered to a hydrophobic backing using an adhesive to form hydrophobic-backed strips. The hydrophobic backed strips may then be cut into cards of test strips and optionally, adhered to a card holder or other type of substrate for supporting the card or test strips.

Advantageously, this method and the automated methods described herein, does not require the application of a hydrophobic agent in order to define the hydrophilic reaction areas. Rather, the layout of the laminated strips onto the hydrophobic backings such that an air gap is present between each laminated strip is sufficient to prevent bleed-over or spillage of a solvent, a reagent or a sample between adjacent laminated strips. The laminated strips and cards/test strips can be cut using any precise cutter, such as an exacto-knife, laser cutter, or craft cutter.

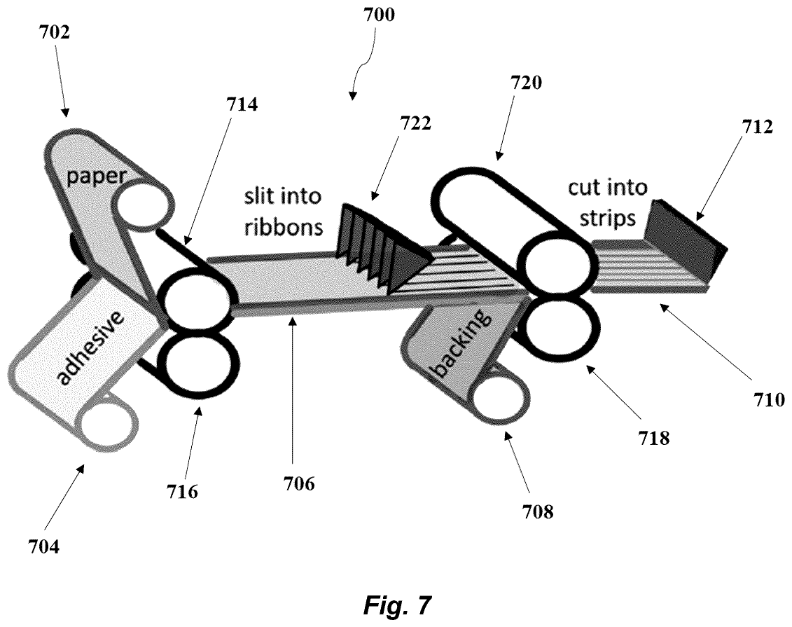

In other embodiments, a method of fabricating a PAD is a semi-automated or fully automated process. FIG. 7 illustrates an embodiment of a roll-to roll method of fabrication a PAD. For example, an apparatus 700 for fabricating a PAD comprises a hydrophilic substrate provided as a first continuous roll 702 and a first double sided adhesive layer provided as a second continuous roll 704. A first laminating roller 714 and a first sub roller 716 laminate the hydrophilic substrate and the double-sided adhesive together to form laminated sheets 706 which are moved by the rollers 714, 716 to cutting device 722. Cutting device 722 may be, for example, a plurality of fixed blades or one or more laser cutting devices. Cutting device 722 cuts the laminated sheets into ribbons of a predetermined width to form laminated strips. The laminated strips are then adhered to a hydrophobic backing provided as a third continuous roll 708. A second laminating roller 720 and a second sub roller 718 laminate the laminated strips to the hydrophobic backing to produce hydrophobic-backed strips. The hydrophobic-backed strips may then be cut into test strips or cards 710 by cutting device 712. Cutting device 712 can be, for example, an actuating cutting blade or a laser cutting device. Optionally, the test strips or card may be affixed to a card holder or other substrate to support said test strips or cards. Assay reagents, fiducial marker, QR codes, and other features of the PADs can then be added to the PAD using methods described herein.

Accordingly, in some embodiments, a method for fabricating a paper analytic device (PAD) comprises i) laminating a porous hydrophilic substrate onto a first double-sided adhesive layer to provide a plurality of laminated sheets; ii) cutting the plurality of laminated sheets lengthwise to provide cut ribbons of laminated strips; iii) laminating one side of the laminated strips with a hydrophobic backing to form hydrophobic-backed strips; wherein each of the laminated strips is positioned on the hydrophobic backing such that each cut ribbon is adjacent to at least one other cut ribbon with an air gap therebetween, wherein the airgap is about 1 mm to about 5 mm; and iv) recutting the hydrophobic-backed strips into test cards of a predefined size.

Some embodiments further comprise the steps of v) applying one or more assay reagent to predefined assay regions of each of the laminated strips; vi) attaching each of the test cards with at least one fiducial marker; and vii) forming a sample deposition area on each of the laminated strips. In some embodiments, the sample deposition area is formed using a solid dosing device or liquid application strip. In some embodiments, the fiducial marker is printed onto the hydrophobic backing. In some embodiments, the sample deposition area is an area of the cut ribbon. In other embodiments, the sample deposition areas may be a predefined region of the cut ribbon or an area cut or etched into the cut ribbon.

In some embodiments, the method may comprise a roll-to-roll process, wherein step i) further comprises one or more of providing a continuous first roll and a continuous second roll, and step iii) further comprises a continuous third roll; wherein the continuous first roll comprises the porous hydrophilic substrate, the continuous second roll comprises the first double-sided adhesive layer, and the third continuous roll comprises the hydrophobic backing.

In some embodiments, the laminated strips are each about 2.5 mm to about 13 mm in width and the air gap barrier is about 1 mm to about 5 mm.

The PADs may be packaged in kits providing a user with all of the materials necessary for using the PAD. For example, the kit may contain solvents, micropipettes, weighing paper, and sample applicator sticks to deposit solid samples to the sample deposition areas. Instructions may be provided as a paper insert within the kit or may be printed on the outside of the kit container. The PADS comprising test reagents may be subjected to degradation due to temperature, light, carbon dioxide, or moisture which may affect the accuracy of the tests performed. As a result, the PADs may be individually packaged and sealed in light- and moisture-resistant packets. Additionally, the packets may be packaged with a desiccant in order to maintain a specific moisture level and remove excess moisture. Typically, the kit includes a PAD as disclosed herein; a solvent sufficient to saturate the paper assay device; and instructions for detecting the presence/absence of a target analyte in a sample, quantifying an analyte in a composition, or measuring the relative amounts of one or more analytes in a composition. In some embodiments, the solvent is one that is sufficient to dissolve or suspend the sample composition containing the analyte to be analyzed. Typically, the kit contains a dish to hold the solvent and a spatula, swab, or pipette for applying the composition onto the PAD.

### Results and Discussion

### PAD Design and Characterization

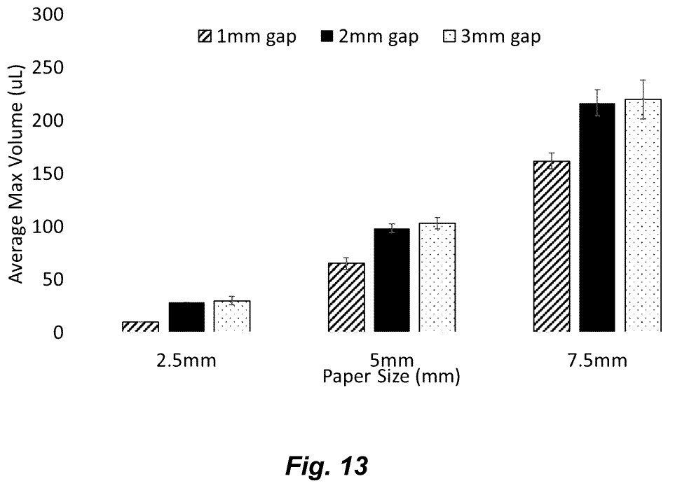

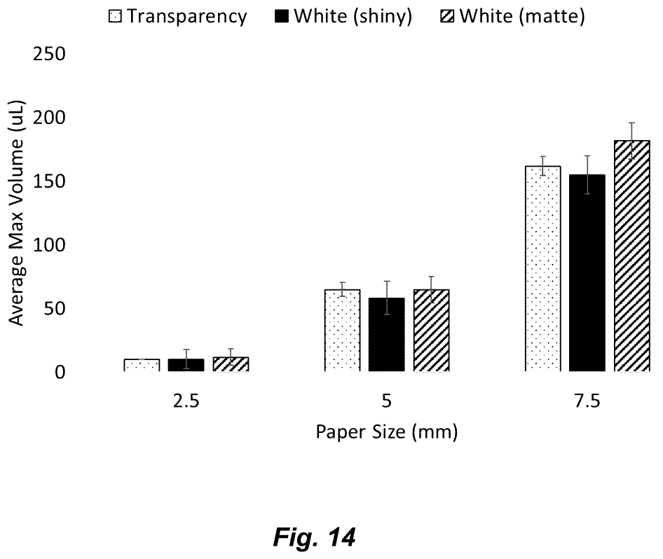

The maximum volume held by a single square of the air-gap device varied linearly with the area of the paper (R2=0.998) with a constant air gap width (FIG. 12). When the air gap width was increased from 1 mm to 2 mm, the maximum volume of all tested paper areas increased (see FIG. 13), but a further increase from 2 mm to 3 mm did not significantly increase the capacity. This was due to the fact that with a small air gap, the paper test zones were so close to each other so that adjacent droplets merged with each other as they grew too large, limiting the maximum volume. With a larger air gap, however, the droplets were farther apart, so the capacity depended only on the area of the paper test zones.

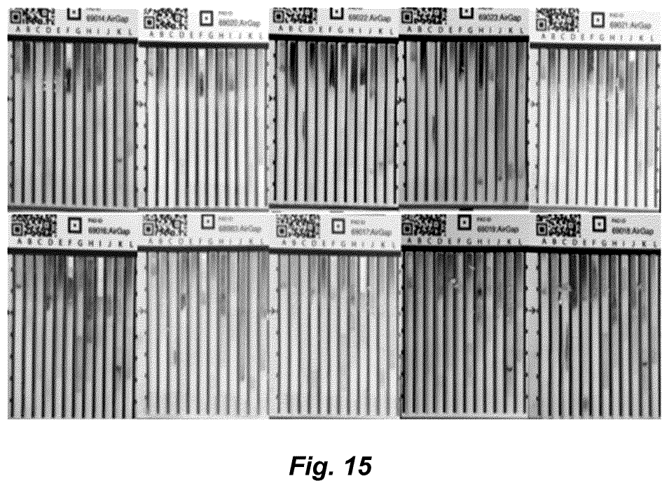

The hydrophobicity of the backing did not significantly affect the maximum volume of the device (see FIG. 14). This is likely due to the water remaining “pinned” to the hydrophilic paper rather than on the backing. In functional use, however, the titration devices with more-hydrophobic backing material were less likely to leak when jostled, so the hydrophobically coated polystyrene film was used for titration experiments and the pilot-scale roll-to-roll production run.

The air-gap barriers successfully contained all tested aqueous solutions, including surfactant solutions, simulated blood, and synthetic urine, but could not contain organic liquids such as ethanol, methanol, isopropanol, acetonitrile, acetone, and hexane.

### Washburn Flow

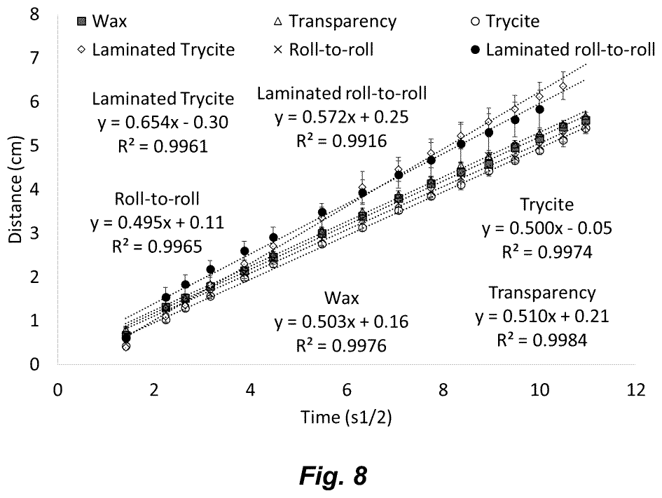

As seen in FIG. 8, the wet-out behavior of the unlaminated air-gap devices was comparable to that of the wax-printed PADs. When the distance traveled by the liquid was plotted against the square root of time according to the Washburn equation, the 5 mm wax-printed devices, transparency-film air-gap devices, plain and laminated hydrophobic Trycite® polystyrene air-gap devices, and 0.2-in plain and laminated roll-to-roll-fabricated devices gave linear graphs (R2=0.9976, 0.9984, 0.9974, 0.9961, 0.9965, and 0.9916 respectively). The slopes of the wax-printed, transparency-film, and Trycite devices were identical within error according to Excel's LINEST function (0.504±0.007 for wax-printed, 0.510±0.006 for transparency-based air-gap, 0.500±0.007 for hydrophobic Trycite® polystyrene-based air-gap, and 0.495±0.008 for roll-to-roll air-gap).

Laminating the air-gap devices with packing tape, however, increased the rate of fluid flow, resulting in a steeper slope (0.655±0.004 for laminated handmade device, 0.572±0.015 for laminated roll-to-roll device). In 100 seconds, the water traveled 6.1 cm up the laminated lanes, but only 4.9 cm up the unlaminated lanes. This is consistent with previous literature reports of faster flow rates in laminated devices. The 7.5 mm lanes of the air-gap and wax-printed devices gave similar slopes to the 5 mm (unlaminated) devices (0.536±0.008 for transparency-based air-gap, 0.498±0.006 for hydrophobic Trycite® polystyrene-based air-gap, and 0.511±0.01 for wax-printed). This suggests that the inherent wetting properties of the paper are not noticeably affected by the differences between the air-gap and wax-printing fabrication methods or by the hydrophobicity of the backing, but if more rapid fluid flow is desired, the device can be laminated.

### Titrator Performance

When a wax-printed titrator and a roll-to-roll air-gap titrator preloaded with p-toluenesulfonic acid were compared side-by-side in a titration with 40 μL of 0.094 M sodium hydroxide analyte and phenolphthalein indicator, the air-gap and wax-printed devices performed virtually identically (see FIG. 9).

Unlike a typical pH-vs.-volume titration curve, where equivalence is found at the inflection point, the endpoint for these titration curves is found at the point where the graph levels off, signaling complete disappearance of the pink color of the phenolphthalein indicator. The exact RGB values obtained by ImageJ analysis were different, as the black wax backing of the wax-printed devices resulted in a darker background color than the white backing of the air-gap devices. Because the air-gap devices do not have interference from the color of the backing, they had less variability in the measured color intensity and therefore smaller error bars. The endpoints, however, were the same for both device types, both by visual inspection and titration curve (FIG. 9).

### 12-Lane PAD Performance

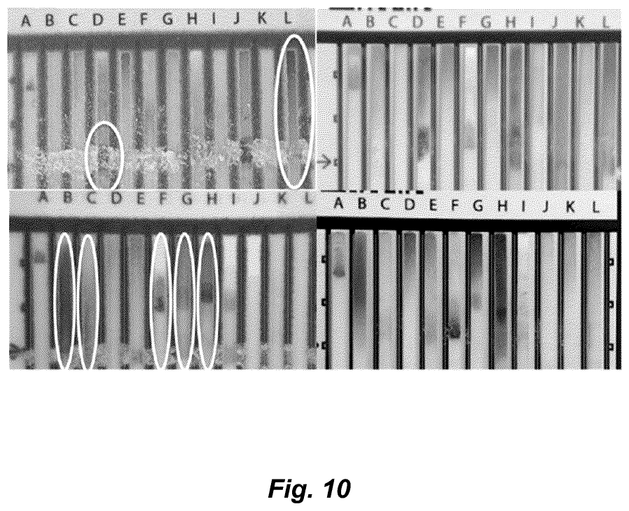

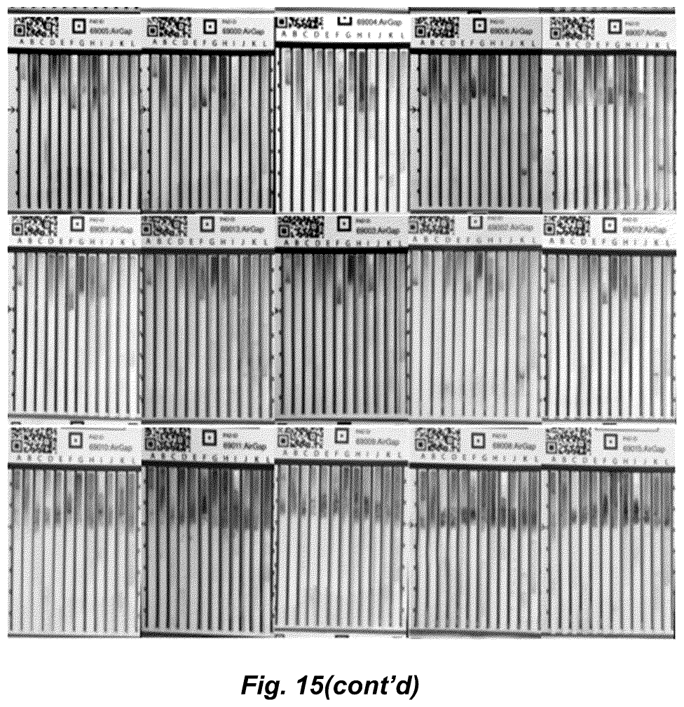

Images of the 12-lane pharmaceutical screening PADs were captured using a mobile app and analyzed using a neural network to identify the active pharmaceutical ingredient as described in Banerjee et al., 2016 IEEE Winter Conference on Applications of Computer Vision, WACV 2016, Institute of Electrical and Electronics Engineers Inc., 2016). The mobile app successfully captured and rectified images of the air-gap PADs (192 images total). Example images of air-gap and wax-printed PADs are shown in FIG. 10; see FIG. 15 for images of all 25 PADs. Neither the app operating system (iOS vs. Android, p-value=0.66) nor the background color (black vs. white, p-value=0.51) significantly affected the accuracy of the neural network (see Table 3 for Student's t-tests).

TABLE 3
Neural network classification accuracy as a function of
mobile app operating system and background color.

| | White | Black | iOS app | Android app |
|---|---|---|---|---|
| | background | background | accuracy | accuracy |
| API | accuracy (%) | accuracy (%) | (%) | (%) |
| Amoxicillin | 63 | 55 | 68 | 47 |
| Ciprofloxacin | 69 | 75 | 69 | 75 |
| Isoniazid | 76 | 85 | 75 | 86 |
| Pyrazinamide | 29 | 68 | 42 | 52 |
| Rifampicin | 70 | 63 | 80 | 55 |
| Average | 61 | 69 | 67 | 62 |
| T-statistic | White vs | 0.72 | iOS vs | 0.48 |
| (df = 4) | black | | Android | |
| P-value | White vs | 0.51 | iOS vs | 0.66 |
| (2-tailed) | black | | Android | |

The neural network struggled to accurately classify the APIs present in the images of the air-gap PADs (64% accuracy for air-gap vs. 98% accuracy for wax-printed; see Table 2). This was not surprising, as the neural network was trained using only wax-printed PAD images. To confirm that the decrease in accuracy was due to the neural network and not to defects in the air-gap PADs themselves, 25 blinded air-gap PAD images (one image for each card) were analyzed by five trained human readers. When the PADs were classified by eye, four out of five readers classified all 25 cards correctly, and the overall average accuracy was 97% (see Table 4).

TABLE 4
Classification accuracy of wax-printed and air-gap
pharmaceutical screening PADs

| | Wax-printed | Air-gap | Air-gap |
|---|---|---|---|
| | accuracy (%) | accuracy (%) | accuracy (%) |
| | (app/neural | (app/neural | (human |
| API | net) | network) | readers) |
| Amoxicillin | 99 | 57 | 100 |
| Ciprofloxacin | 92 | 72 | 100 |
| Isoniazid | 100 | 80 | 100 |
| Pyrazinamide | 100 | 45 | 88 |
| Rifampicin | 100 | 68 | 96 |
| Average ± | 98 ± 4 | 64 ± 14 | 97 ± 5 |
| Standard | | | |
| Deviation | | | |

For both human readers and the cell-phone app, pyrazinamide was the most difficult drug of the five APIs studied to classify correctly, most likely because its “color barcode” consists of only one distinct color change (a dark red color in lane G), which can be easy to overlook. Isoniazid, in contrast, gives distinct color changes in five lanes, which makes it easier to classify accurately. One potential reason for amoxicillin's low accuracy on the app but high accuracy with human readers is that one of its three color changes (the red “flame” in lane K) can be unreliable if the PAD has been stored for several months. A human reader can still reliably identify amoxicillin based on the other two predicted color changes, but a neural network may struggle. Another potential explanation for the differences in accuracy between the app and human readers is the fact that the paper strips in the prototype air-gap PADs had a small amount of browning from the laser-cutting process, which altered the shade of the colors slightly (as the roll-to-roll process uses knives rather than a laser cutter, this is not an issue for scaled-up air-gap manufacturing). For a neural network, this alteration in color intensity is problematic, but a human reader can still readily identify the colors.

Our results, then, show that the air-gap PAD performed comparably to the wax-printed PAD when analyzed by human readers. Since the PADs were originally developed to be read by eye and can be reliably used without the cell-phone app and neural network (Eberle et al., J Glob Oncol, 2020, 6, 407-413; Bliese et al., Analytical Methods, 2019, 11, 4727-4732; Chikowe et al., Am J Trop Med Hyg, 2018, 99, 233-23 8), this shows that the air-gap PADs are a viable successor to the wax-printed 12-lane PADs. Our challenges when attempting to analyze the air-gap PADs with a neural network designed for wax-printed PADs, however, highlight the fact that changing external factors, like the fabrication method, will likely necessitate retraining any neural networks used for computer-assisted classification. We anticipate that retraining our neural network on images of air-gap PADs will ameliorate many of these issues and improve the accuracy of the cell-phone app's classification.

### Roll-to-Roll Production Run

We partnered with a local test-strip manufacturer to perform a pilot roll-to-roll production run of the air-gap devices. Because we use a 96-well inoculator or a multichannel pipette to deposit reagents, the paper lanes needed to be spaced at 9 mm, and thus two paper guides were custom machined ($3400, one-time cost) with this spacing. Excluding the one-time cost of the guides, the total cost for materials, equipment use, and labor was —$4500. A 500 ft roll of 8-in paper produced approximately 2700 feet of assembled cards, which translates to either 10,800 3-in cards for pharmaceutical screening or 162,000 ⅕-in test strips for paper-based titrations. This brings the cost per device to $0.41 per 3-in PAD ($0.73, including one-time costs) or $0.026 per ⅕-in titrator ($0.05, including one-time costs).

Our manufacturing partner's current equipment is limited to a maximum of seven paper lanes no narrower than 0.2 inches. The current 7-lane PADs are useful for the paper titrators (Roller et al., J Chem Educ, 2021, 98, 1946-1953) and a pared-down version of the PAD used for illicit drug analysis, but in future production runs, we plan to obtain a knife set capable of cutting 2.5 mm lanes, as well as another custom guide that can deposit 12 paper strips at a time so we can mass produce the 12-lane pharmaceutical PADs.

The air-gap design offers a simple, scalable alternative to traditional methods of fabricating paper microfluidics. Air-gap devices can be readily fabricated by hand for prototyping and device development, but more importantly, we have shown that they can be mass produced with roll-to-roll manufacturing. Since the necessary roll-to-roll equipment is commonly available at many test-strip manufacturers, it is possible to partner with a company to produce air-gap devices at scale without purchasing manufacturing equipment. At the pilot-manufacturing scale, the cost of air-gap device fabrication was as low as $0.03 per device, including labor, equipment use, and raw materials.

This roll-to-roll method can create PADs consisting of straight paper channels and square dot features. Further development will be needed to create more general designs with curved lines, holes, and complex shapes, or to incorporate folded, rolled, or stacked multi-level structures which are readily accessible by other fabrication methods. The current method, however, is applicable not only to our two device designs, the paper titrator and the 12-lane PAD, but also to the many PADs reported by other groups that involve spot tests and/or straight channels.

Our testing showed that air-gap devices performed comparably with wax-printed devices for paper-based titrations and pharmaceutical screening. Future research will focus on training our neural network to recognize air-gap pharmaceutical screening devices and expanding the air-gap method to other device architectures.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

### PAD Fabrication

Air-gap PADs can be assembled by hand for prototyping, or mass produced using a roll-to-roll method.

Hand fabrication. To fabricate the air-gap devices by hand for prototyping purposes, Ahlstrom 319 fast chromatography paper (Midland Scientific, Chicago, IL) was backed with double-sided pressure-sensitive adhesive (Artgrafix, Beacon Falls, CT) and cut into strips with a Glowforge Basic laser cutter. These strips were mounted on a plastic backing using a pegboard for alignment. The assembly was then sliced crossways into air-gap devices (see FIG. 6).

Roll-to-roll production. For the roll-to-roll manufacturing, we collaborated with Serim Research Corporation, a test-strip manufacturer in Elkhart, IN. For this method, a 500 ft roll of 8-inch-wide Ahlstrom 319 paper was dry-laminated with double-sided adhesive (3M double-coated tape 415, 3M, Saint Paul, MN) and slit into 0.2-inch ribbons (i.e., laminated strips). Seven of these ribbons (laminated strips) were laminated onto a roll of 3.25-inch-wide white, hydrophobically coated polystyrene (Trycite, Franklin Park, Ill., lot #1345300) with 9 mm spacing and cut crossways into 9-inch cards. These 9-inch cards were further cut into either 3-inch cards or 0.2-inch strips. Before cutting, the back of the polystyrene was laminated with double-sided adhesive so that test strips could be attached to custom-printed card holders. (See FIG. 7)

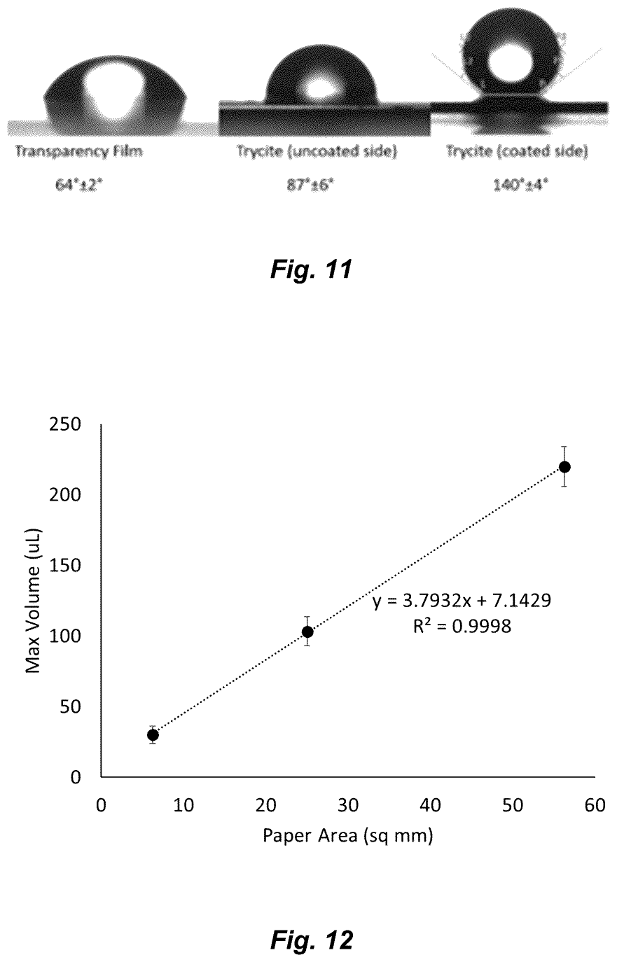

PAD characterization and design considerations. Three different backing types with varying degrees of hydrophobicity were assessed for their performance in the air-gap devices: commercial overhead transparency film (C-line, Mt Prospect, IL) and both the coated and uncoated sides of Trycite® polystyrene film (Transcendia, Franklin Park, Ill.). Measurement with a contact angle goniometer (DropMaster DMo-701, Kyowa Interface Science Co., Japan) using the sessile drop method on nine replicates, analyzed using the tangent method in FAMAS software, showed that the transparency film was moderately hydrophilic (θ=64°±2°, the uncoated side of the white film was on the border between hydrophobic and hydrophilic (θ=87°±6°, and the coated side of the white film was strongly hydrophobic (θ=)140°±4° (see FIG. 11).

Three design variables were considered for their effect on device volume: paper area, air gap width, and backing hydrophobicity. Paper squares measuring 2.5×2.5 mm, 5×5 mm, and 7.5×7.5 mm were placed on the three backing types (transparency film, coated and uncoated polystyrene) with air gaps of 1-, 2-, and 3-mm. Deionized water was added to two adjacent paper squares in 10 μL increments until the surface tension broke or the adjacent droplets merged.

Air-gap devices were also tested for their ability to contain other liquids, including simulated blood (Type A, Ward's Science, VWR, St. Catherine, Ontario), synthetic urine (RICCA Chemical Company, Arlington, TX), TWEEN® 20 (Sigma-Aldrich) surfactant solutions (2%, 5%, 10%, 20%, and 50%), ethanol, methanol, isopropanol, acetonitrile, acetone, and hexanes.

Paper

Washburn flow. To compare the wet-out behavior of the air-gap devices to that of wax-printed μPADs, air-gap devices (2.5, 5, and 7.5 mm strips on moderately hydrophilic transparency film or on hydrophobic Trycite® polystyrene film) and wax-printed devices (2.5, 5, and 7.5 mm paper lanes separated with wax barriers, 1 mm before baking, 1.25 mm after baking) were placed on end in 1 cm deionized water tinted with blue food coloring (FD&C Blue 1, McCormick, Duluth, GA) and filmed for two minutes. To study the effect of lamination on the air-gap PADs, devices were laminated with clear packing tape (Scotch Heavy Duty Packing Tape, Office Depot) to within 1 cm of the bottom of the PAD. Video frames were analyzed at 1, 2, 5, and 10 seconds and at 10-second intervals thereafter to track the flow of liquid up the paper lanes. Wet-out behavior was then quantified using the Washburn equation:

$$L = \sqrt{\frac{γ r cos ( θ )}{2 η} t}$$

which models the distance L traveled by a liquid with surface tension γ and viscosity η through a medium with pore radius r and contact angle θ in time t. Twelve replicate measurements were taken of each type of device.

### Titrator Testing

The air-gap device's performance as a vehicle for paper-based titrations was compared to the wax-printed titrator device described by Roller et al., J Chem Educ, 2021, 98, 1946-1953. Both the wax-printed and air-gap titrators were pre-loaded with 5 μL per square of p-toluenesulfonic acid (Alpha Aesar, Ward Hill, MA) solutions ranging from 100 mM to 3 M (see FIG. 9 below). 40 μL of 0.094 M sodium hydroxide (Fisher Scientific, Fair Lawn, NJ) with phenolphthalein indicator (HiMedia Laboratories, Dindori, India, 1-2 drops of 5% indicator solution per 10 mL analyte) was then added to each square to perform the limit titration. Six replicates of each device type were imaged and analyzed in ImageJ to obtain titration curves. The green channel (G) was chosen for the titration curves as it is a close proxy for measuring the absorbance of the pink phenolphthalein solutions.

### 12-Lane PAD Testing

To compare the air-gap device's potential as an alternative to the wax-printed 12-lane PAD developed by the Lieberman lab for pharmaceutical analysis, air-gap PADs were fabricated (2.5 mm-wide laser-cut paper strips spaced 2 mm apart on transparency film) and stamped with the reagents specified by Bliese et al., Anal. Methods, 2019, 11, 4727-4732. Five active pharmaceutical ingredients (APIs) were tested: amoxicillin, ciprofloxacin, isoniazid, pyrazinamide, and rifampicin. Five PADs were run for each API. Images of the PADs were captured, rectified, and classified by a neural network (Banerjee et al.) using a cell phone app.(see www.paperanalytics.org). For image capture by the app, transparency-film-based air-gap PADs were placed atop a piece of paper printed with the fiducial markings normally printed on the PAD itself. (see Banerjee et al.) The fiducials allow the app to recognize, align, and rectify the captured image. Each PAD was imaged against both dark and white background with four different devices—an iPhone, an iPad, a Google Pixel, and a Nokia phone—to obtain a total of 200 images. Blinded images of all 25 PADs (one image per PAD) were also read by eye by five trained users.

### Example 2. A 12-Lane PAD for Detecting Pharmaceutical Analytes

A 12-lane PAD for detecting pharmaceutical such as, but not limited to, albendazole, amoxicillin, ampicillin, azithromycin, benzylpenicillin, ceftriaxone, chloroquine, ciprofloxacin, doxycycline, epinephrine, ethambutol, ferrous sulfate, hydroxychloroquine, isoniazid, promethazine hydrochloride, pyrazinamide, rifampicin, sulfamethoxazole, tetracycline, and a mixture of rifampicin/isoniazid/pyrazinamide/ethambutol (RIPE). Table 1 describes the reagent layout with reference to lane, row, and reagents deposited in specific row to detect pharmaceuticals.

Expected color signature indicating a sample positive for the specific analyte (i.e., a pharmaceutical): amoxicillin: lane B: olive green, lane F: dark green/black, lane K: red; ciprofloxacin: lane D: blue at swipe line, lane L: orange; pyrazinamide: lane G: red; rifampicin: lane C: reddish brown, lane F:orange to black, lane G: reddish purple brown, lane K: orange, lane L: purple gray; isoniazid: lane B: orange, lane C: yellowish green, lane F: green spot, lane G: orange; lane H: orange/red; azithromycin: lane D: blue at swipe line; ceftriaxone: lane C: green, lane F: olive green, lane G: orange, lane H: purple-brown, lane K: dark red on top of red, lane L: orange-red; chloroquine: lane D and E: bright blue at swipe line; hydroxychloroquine: lanes D and E: spotty deep blue; doxycycline: lane F: yellowish to green to blue up the lane, lane L: brown; albendazole: lane D: blue at swipe line; benzylpenicillin: lane D: slight blue at swipe line, lane E: slight purple at swipe line, lane F: black spot; ampicillin: lane B: light orange, lane C: purple-brown, lane F: black spot, lane I: dark gray, epinephrine: lane C: yellowish green, lane E: brown, lane I: brown, and L: black; ethambutol: lane B: light orange; ferrous sulfate: lanes A, B, D, F, G, and K: brown at swipe line, lane I: brown at the top; promethazine hydrochloride: lanes C and D: bright blue at swipe line, lanes G and K: orange at swipe line; sulfamethoxazole: lane K: orange; tetracycline: lane C: green, lane F: yellow to blue, lane I: green, lane K: red-brown, lane K: brown; RIPE (rifampicin, isoniazid, pyrazinamide, ethambutol mixture): lane C: brown to blue, lane F: orange to brown to blue, lanes G and H: dark red spot, lane I: brown at swipe line, lane K: orange, lane L: purple-brown. The colors are compared to a negative control without a sample (water used as mobile solvent).

### Example 3. Six and Twelve Lane PAD for Detecting Illicit Drugs

Exemplary six and twelve lane PADs for detecting illicit drugs such as, but not limited to, cocaine, cocaine HCl, methamphetamine, heroin, and (MDMA). Table 4 and 5 describes the reagent layout with reference to lane, row, and reagents deposited in specific row to detect illicit drugs.

TABLE 4
Reagent layout for illicit drugs. Six lane PAD.

| | | | Tests |
|---|---|---|---|
| Lane | Row | Reagent | for: |
| A | 4 | [Co(SCN)₄]²⁻ 1M | tertiary |
| | 5 | [Co(SCN)₄]²⁻ 1M | amines |
| | 6 | Tosic acid(1M) | |
| | 7 | Tosic acid (1M) | |
| B | 4 | [Co(SCN)₄]²⁻ 1M | tertiary |
| | 5 | [Co(SCN)₄]²⁻ 1M | amines |
| | 6 | NaOH (2M) | |
| | 7 | NaOH (2M) | |
| C | 2 | NaOH (2M) | phenol |
| | 3 | NaOH (2M) | |
| | 4 | Tosic acid (1M) | |
| | 6 | p-nitroaniline | |
| | | (0.2M) (light | |
| | | orange color) | |
| | 7 | NaNO₂ (0.4M) | |
| D | 2 | NaOH (2M) | phenol |
| | 3 | NaOH (2M) | |
| | 4 | Tosic acid (1M) | |
| | 6 | Sulfanilamide | |
| | | (0.17M) | |
| | 7 | NaNO₂ (0.4M) | |
| E | 2 | NaOH (4M) | aniline |
| | 3 | NaOH (2M) | |
| | 5 | NQS (0.03M) | |
| | | (yellow/orange | |
| | | color) | |
| | 6 | Tosic acid (1M) | |
| F | 3 | DMAC | Indoles |
| | 4 | DMAC | |
| | 6 | Tosic acid(1M) | |
| | 7 | Tosic acid (1M) | |

TABLE 5
Reagent layout for illicit drugs. Twelve lane PAD.

| Lane | Row | Test | Reagent |
|---|---|---|---|
| A | 3/4 | Acidic cobalt | 1M: 3 g Co(II) nitrate hexahydrate + |
| | | thiocyanate | 2 g KSCN in 10 mL H₂O) |
| | 5/6 | | 1M: 1.76 g tosic acid in 10 ml H₂O |
| B | 3/4 | pH 8 cobalt | 1M: 1M: 3 g Co(II) nitrate hexahydrate + |
| | | thiocyanate | 2 g KSCN in 10 mL H₂O) |
| | 5/6 | | 1M tris base and 1M TrisHCL mixed to pH |
| C | 3/4 | Basic Co(SCN)₂ | 1M: 3 g Co(II) nitrate hexahydrate + |
| | | | 2 g KSCN in 10 mL H₂O) |
| | 5/6 | | 2M: 0.8 g NaOH in 10 mL of H₂O |
| D | 1/2 | P-Nitroaniline | 2M: 0.8 g NaOH in 10 mL of H₂O |
| | 3 | | 1M: 1.76 g tosic acid in 10 ml H₂O |
| | 5 | | 0.2M: 0.3 g sulfanilimide in 10 mL H₂O |
| | 6 | | 0.4M: 0.3 g NaNO2 in 10 mL H₂O |
| E | 1/2 | Sulfanilamide | 2M: 0.8 g NaOH in 10 mL of H₂O |
| | 3 | | 1M: 1.76 g tosic acid in 10 ml H₂O |
| | 5 | | 0.17M: 0.3 g sulfanilamide in 10 mL H₂O |
| | 6 | | 0.4M: 0.3 g NaNO2 in 10 mL H₂O |
| F | 1 | NQS (Nu) | 4M: 1.6 g NaOH in 10 mL H₂O |
| | 2 | | 2M: 0.8 g NaOH in 10 mL H₂O |
| | 3 | | 0.03M: 0.1 g napthoquinine sulfonate |
| | | | in 10 mL H₂O |
| | 4 | | 1M: 1.76 g tosic acid in 10 ml H₂O |
| G | 3 | Starch | 0.4 g povidone in 10 mL H₂O (4% PVD) |
| | 4 | | 0.05 g I₂ and 0.1 g KI in 10 mL H₂O |
| H | 2 | Ethambutol | 2M: 0.8 g NaOH in 10 mL H₂O |
| | 3 | | 1M: 2.5 g CuSO₄ in 10 mL H₂O |
| I | 3/4 | Carbonate | 0.3M: 0.5 g FeCl₃ in 10 mL H₂O |
| J | 2/3 | Ninhydrin | 0.17M: 0.4 g Ninhydrin in 10 mL CH₃CN |
| | 4/5 | | 2M: 2.76 g Ninhydrin in 10 mL H₂O |
| K | 2/3 | DMAC | 0.1 g dimethylaminocinnamaldehyde |
| | | | in 10 mL CH₃CN |
| | 5/6 | | 1M: 1.76 g tosic acid in 10 ml H₂O |
| L | 0 | Timer | 0.2M: 0.713 g NiCl₂ in 15 mL H₂O |
| | 3 | | 0.2M 0.3 g DMG in 10 mL 0.4M NaOH |

Expected color signature indicating a sample positive for the specific analyte (i.e., an illicit drug or adulterant): Heroin: Lanes A, B, and C blue; lanes D and E brown/red. Cocaine HCl: Lanes A, B, and C teal blue. Crack cocaine: lanes A and C blue. Methamphetamine: Lanes A, B, and C blue but with central part of the lane “washed out,” lane F yellow to red. MDMA: lanes A, B, and C blue; lane F yellow to red. Tramadol: lanes A, B, and C blue. Acetaminophen: lane D yellow to brown, lane E reddish brown. Procaine: lanes D and E brown, lane K deep purple. Dapsone: lane F pink/red, lane K black. Epinephrine: lanes F and H dark brown, lane I dark teal. Starch: lane G black at swipe line. Metformin: lane H pink to purple to blue. Benzocaine: lane K bright purple.

### Example 4. A 12-Lane PAD for Detecting Chemotherapeutic Agents

A 12-lane PAD for detecting chemotherapeutic agents such as, but not limited to, cisplatin, oxaliplatin, methotrexate, doxorubicin, and sodium 2-mercaptoethane sulfonate (MESNA). Table 6 describes the reagent layout with reference to lane, row, and reagents deposited in specific row to detect chemotherapeutic agents.

TABLE 6
Reagent layout for chemotherapeutic agents.

| Test | Lane | Row | Reagent |
|---|---|---|---|
| Timer | A | 0 | NiCl₂ 0.20M |
| | | 3 | Dimethyl glyoxime |
| | | | DMG 0.20M |
| Ninhydrin | B | 2 | Ninhydrin 30 mg/mL |
| | | 3 | Ninhydrin 30 mg/mL |
| | | 4 | K₂CO₃ 2M |
| | | 5 | K₂CO₃ 2M |
| Tin Chloride | C | 2 | SnCl₂ |
| | | 3 | SnCl₂ |
| Tin Chloride + | D | 2 | SnCl₂ + PEG |
| PEG | | 3 | SnCl₂ + PEG |
| pH 8 Co(SCN)₂ | E | 3 | Co(SCN)₂ 1M |
| | | 4 | Co(SCN)₂ 1M |
| | | 5 | Tris Buffer pH 8 1M |
| | | 6 | Tris Buffer pH 8 1M |
| Beta Lactum | F | 1 | CuSO₄*5H₂O 1M |
| | | 2 | K2CO3 2M |
| Vanadium | G | 2 | NaOH 1M |
| | | 3 | VOSO₄ |
| | | 4 | VOSO₄ |
| 4-Naphthaquinone | H | 1 | NaOH 4 M |
| sulfonate (NQS) | | 3 | NQS 10 mg/mL |
| | | 4 | NQS 10 mg/mL |
| Ethambutol | I | 1 | NaOH 2M |
| | | 3 | CuSO₄*5H₂O 1M |
| Starch | J | 3 | Triiodine and 2% Povidone |
| | | 4 | Triiodine and 2% Povidone |
| Zinc Chloride | K | 2 | NaOH 1M |
| | | 3 | ZnCl₂ |
| | | 4 | ZnCl₂ |
| Carbonate | L | 3 | FeCl₃ 50 mg/mL |
| | | 4 | FeCl₃ 50 mg/mL |

Expected color signature indicating a sample positive for the specific analyte (i.e., a chemotherapeutic drug): Cisplatin (dosage form is 1 mg/mL): Lanes C and D yellow. Oxaliplatin (dosage form is 5 mg/mL): Lanes C and D orange. Intensity of color varies with concentration of platinum drug. Doxorubicin: Pink color across all the swipe lines in all the lanes; black color in lane L. Methotrexate: Yellow at top of lane A, green in lane F. MESNA: lane E dark brown.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and in particular, Smith et al., J Glob Oncol. 2018 Dec.; 4:1-10., U.S. Pat. Nos. 9,354,181 and 9,557,274 to Barstis et al., and patent documents U.S. Pat. Pub. No 2015/0160245 to Lieberman et al. are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A paper analytical device (PAD) comprising:
one or more laminated strips comprising a porous hydrophilic substrate and a first adhesive layer, wherein the one or more laminated strips are disposed on hydrophobic backing with an air gap barrier between each of the one or more laminated strips disposed on the hydrophobic backing, wherein the air gap barrier is about 1 mm to about 5 mm in width and extends along an entire length of the one or more laminated strip;
one or more assay regions disposed on the one or more laminated strips wherein the one or more assay regions comprises one or more reagents for detecting an analyte; and
one or more sample deposition areas on the one or more laminated strips, wherein the PAD is activated when one or more solvents travel through both the one or more sample deposition areas and the one or more assay regions due to capillary action.

2. The PAD of claim 1, further comprising a second adhesive layer laminated to the hydrophobic backing.

3. The PAD of claim 2, wherein the PAD is disposed on a print card of a defined width and length.

4. The PAD of claim 1, further comprising at least one optically readable information area that provides visual information for detection of analyte,
wherein the at least one optically readable information area comprises one or more alignment references for transforming or correcting a captured image of the PAD to facilitate analysis.

5. The PAD of claim 4, wherein the one or more alignment references include a plurality of fiducial markers for orienting a captured image of the PAD, and the PAD further comprises an identification tag of a two-dimensional barcode.

6. The PAD of claim 1, wherein a width of a laminated strip is about 2 mm to about 7 mm; and
a width of the air gap barrier is about 2 mm to about 4.5 mm in width.

7. The PAD of claim 4, wherein the visual information comprises color information caused by an interaction of the analyte and the one or more reagents.

8. The PAD of claim 1, wherein the analyte comprises one or more of an antimicrobial agent, a chemotherapeutic agent, and an illicit drug.

9. The PAD of claim 8, wherein the antimicrobial agent comprises one or more of albendazole, amoxicillin, ampicillin, azithromycin, benzylpenicillin, ceftriaxone, chloroquine, ciprofloxacin, doxycycline, epinephrine, ethambutol, ferrous sulfate, hydroxychloroquine, isoniazid, promethazine hydrochloride, pyrazinamide, rifampicin, sulfamethoxazole, tetracycline, and a mixture thereof;
the chemotherapeutic agent comprises one or more of cisplatin, oxaliplatin, methotrexate, sodium 2-mercaptoethane sulfonate (MESNA) and doxycycline; and
the illicit drug comprises one or more of an opiate or synthetic opioid, cocaine, 3,4-methylenedioxymethamphetamine (MDMA), and methamphetamine.

10. The PAD of claim 1, wherein the analyte comprises a solid powder applied directly to the one or more sample deposition areas.

11. A method for fabricating a paper analytic device (PAD) comprising:
i) laminating a porous hydrophilic substrate onto a first double-sided adhesive layer to provide a plurality of laminated sheets;
ii) cutting the plurality of laminated sheets lengthwise to provide cut ribbons of laminated strips;
iii) laminating one side of the laminated strips with a hydrophobic backing to form hydrophobic-backed strips; wherein each of the laminated strips is positioned on the hydrophobic backing such that each cut ribbon is adjacent to at least one other cut ribbon with an air gap therebetween, wherein the airgap is about 1 mm to about 5 mm; and
iv) recutting the hydrophobic-backed strips into test cards of a predefined size.

12. The method of claim 11, further comprising:
v) applying one or more assay reagent to predefined assay regions of each of the laminated strips;
vi) attaching or printing at least one fiducial marker to each of the test cards; and
vii) forming a sample deposition area on each of the laminated strips, wherein the sample deposition area is formed using a solid dosing device or liquid application strip.

13. The method of claim 12, wherein the method comprises a roll-to-roll process, wherein step i) further comprises one or more of providing a continuous first roll and a continuous second roll, and step iii) further comprises a continuous third roll;
wherein the continuous first roll comprises the porous hydrophilic substrate, the continuous second roll comprises the first double-sided adhesive layer, and the third continuous roll comprises the hydrophobic backing.

14. The method of claim 10, wherein the laminated strips are each about 2.5 mm to about 13 mm in width.

15. The method of claim 10, wherein the airgap is about 1 mm to about 5 mm.

16. A method for detecting an analyte in a sample comprising:
depositing one or more samples comprising an analyte in the one or more sample deposition areas of the porous hydrophilic substrate of the PAD of claim 1, wherein at least one of the one or more samples comprises a solid powder applied directly to at least one of the one or more sample deposition areas;
activating the PAD by contact with a solvent to permit the sample to flow by capillary action through the one or more assay regions containing the one or more reagents for detecting the analyte to the one or more sample deposition areas so that the one or more assay reagents for detecting the analyte and the sample come into contact to provide visual information for analysis; and
analyzing the visual information to detect the presence or the absence of the analyte in the sample.

17. The method of claim 16, wherein providing the visual information for analysis comprises causing a color change that is analyzed to detect the presence or the absence of the analyte in the sample when the one or more assay reagents for detecting the analyte and the sample come into contact.

18. The method of claim 16, further comprising automating the visual information for analysis by:
capturing an image of the PAD using a camera device; and
using image analysis software configured to recognize and quantify a color change within the one or more assay regions of the PAD shown in the captured image as compared to an image of a reference standard stored in a reference database.

19. The method of claim 19, wherein the PAD further comprises a plurality of fiducial markers for orienting the captured image; and
i) correcting and/or transforming the captured image, by the image analysis software, based on the fiducial marks to align the captured image with the stored image of the reference standard in the reference database;
ii) reading the color change, by the image analysis software, from pre-specified regions in the corrected and/or transformed image; and
iii) determining, by the image analysis software, the presence or the absence of the analyte based on the comparison of the color changes to the stored reference standard images in the reference database.

20. The method of claim 16, wherein the depositing of the one or more samples in the one or more sample deposition areas comprises a use of:
i) an applicator strip for liquid samples; or
ii) a dosing device for solid samples, wherein the dosing device comprises an overlay template comprising one or more windows of a predefined size, wherein each of the one or more windows is configured to align with the one or more sample deposition areas, and wherein the predefined size controls an amount of the one or more samples deposited in the one or more sample deposition areas.
